(19) United States
(12) Patent Application Publication
(10) Pub. No.: US 2004/0156742 A1
(43) Pub. Date: Aug. 12, 2004

(54) Synergistically-effective cyclohexylethan-1-yl ester mixtures as malodour counteractants as measured physiologically and psychometrically and methods for using same

(76) Inventors: Jolanda Bianca Milan, Amsterdam (NL); Simon O'Connor, Cambridge (GB); Tim J.C. Jacob, Cardiff (GB); Virgil A.G. Williams, Leonardo, NJ (US); Stephen P. Nicoll, Ramsey, NJ (US); Richard M. Boden, Ocean, NJ (US); Liwei Wang, Cardiff (GB)

Correspondence Address: Joseph F. Leightner, Esq., INTERNATIONAL FLAVORS & FRAGRANCES INC., 521 West 57th Street, New York, NY, 10019, US

(21) Appl. No.: 10/364,234

(22) Filed: Feb. 11, 2003

(51) Int. Cl. A61L 9/00; B32B 5/02

(52) U.S. Cl. 422/4; 422/5; 422/120; 422/123

(57) ABSTRACT

Described is an ester mixture of 1-cyclohexylethan-1-yl butyrate and 1-cyclohexylethan-1-yl acetate wherein the weight ratio of 1-cyclohexylethan-1-yl butyrate:1-cyclohexylethan-1-yl acetate is from about 20:80 up to about 80:20. The mixture is synergistically effective for its ability to counteract a malodour (1) emanating from a malodourous solid or liquid source into a 3-space proximate the solid or liquid source or (2) present in a malodourous air 3-space.

### FIELD OF THE INVENTION

Our invention is directed to an ester mixture consisting essentially of 1-cyclohexylethan-1-yl butyrate having the structure:

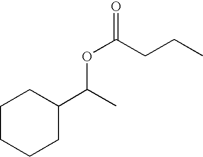

and 1-cyclohexylethan-1-yl acetate having the structure:

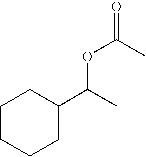

the weight ratio of 1-cyclohexylethan-1-yl butyrate: 1-cyclohexylethan-1-yl acetate being from about 20:80 up to about 80:20, in the substantial absence of the compounds:

1-cyclohexylethan-1-ol having the structure:

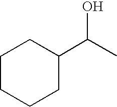

1-(4′-methylethyl)cyclohexylethan-1-yl propionate having the structure:

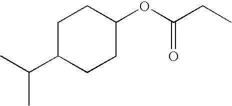

and 2′-hydroxy-1′-ethyl(2-phenoxy)acetate having the structure:

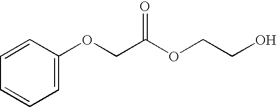

or in the substantial absence of any additional fragrance substances or malodour counteractant substances. The ester mixture is synergistically effective for its ability to counteract a malodour (1) emanating from a malodourous solid or liquid source into a 3-space proximate the solid or liquid source or (2) present in a malodourous air 3-space. The malodourous solid or liquid source and the malodourous air 3-space may or may not contain an additional desirable fragrance. The ester mixture is used as a malodour counteractant by introducing into the 3-space containing the malodour, or proximate the solid or liquid malodourous source an effective malodour-counteracting quantity and concentration of the ester mixture as a single dose, as a continuous dose over a malodour-counteracting period of time, or as periodic doses over a malodour-counteracting period of time whereby the perceived total malodour intensity is substantially reduced or eliminated. When the 3-space contains, in addition to the malodour, a desirable fragrance, the addition of an effective malodour-counteracting quantity and concentration of the synergistically-effective ester mixture as a single dose, as a continuous dose over a malodour-counteracting period of time, or as periodic doses over a malodour-counteracting period of time to the 3-space does not eliminate or substantially reduce the perceived odour intensity of the desired fragrance. The term “3-space” is herein intended to mean “3-dimensional volume, as measured using “x”, “y” and “z” coordinates”.

### BACKGROUND OF THE INVENTION

A wide variety of solid, liquid and gaseous functional materials including body deodorants, antiperspirants, anti-perspirant/body deodorant devices, air fresheners which include air freshening devices, and solid and liquid air freshening and room freshening compositions, room deodorants, herbicides, antiviral compositions, fungicides, bactericides, parasiticides, insecticides, depilatory compositions, bleach compositions, hard surface-cleaning compositions, skin cleansing compositions, antimicrobial nail preparations including anti-fungal nail lacquers, hair setting compositions, hair conditioning compositions, trichological lotions, detergent compositions, soap compositions, sunscreen compositions, fabric stain-removal compositions, fabric conditioning compositions, fabric anti-wrinkle compositions, skin lightening compositions, steam iron aroma compositions including stress relief compositions, candle compositions, plant growth regulating compositions, plant growth stimulating compositions, fertilizer compositions, insect attractant compositions, insect repelling compositions, drain cleaning compositions and molluskicide compositions have been developed that, although useful for their respective purposes, on use thereof emanate odours which are offensive to the human sense of smell. In addition, a number of defined 3-spaces the use of which is required for various business and service operations and personal matters including indoor gymnasiums, indoor sporting event arenas, locker rooms, hair salons, nail salons, tanning salons, beauty salons, tattoo parlors, pig pens, chicken coops, cow barn enclosures, horse barn enclosures, indoor fresh fish markets, plant processing factory rooms, clothing dry cleaning rooms, garment laundry interiors, rooms containing in-use animal litter containers, abattoirs, cattle cars, zoo animal pens, morgues, autopsy rooms, lavatories, medical patient care rooms, hospital wards and dental patient care rooms have continuously prevailing odours which are offensive to the human sense of smell. Such odors which are offensive to the human sense of smell are caused by aliphatic halohydrins, aliphatic amines, aliphatic N-oxides, dialkylamines, cycloaliphatic amines, cycloaliphatic N-oxides, cyclo-olefinic amines, cyclo-olefinic N-oxides, cycloaromatic amines, cycloaromatic N-oxides, hydroxyalkylamines, imine compounds, amide compounds, amino acids, polypeptides, modified antimicrobial proteins, diureides, nitriles, aliphatic mercaptans, cycloaliphatic mercaptans, mercaptoalkanoic acids, mercaptoalkanoic acid esters, aliphatic monosulfides, disulfides, trisulfides, sulfur oxides, sulfones and sultones, cycloaliphatic monosulfides, disulfides, trisulfides, sulfur oxides, sulfones and sultones, cyclo-olefinic monosulfides, disulfides, trisulfides, sulfur oxides, sulfones and sultones, cycloaromatic monosulfides, disulfides, trisulfides, sulfur oxides, sulfones and sultones, alkali metal sulfites, bisulffites and metabisulfites, isothiocyanates, thiocyanates, dithiocyanates, isothiazolones, isothiazolinones, thiodiazinethiones, halosulfamates, aryl sulfonamides, lower aliphatic carboxylic acids, phenols, phosphines, aliphatic phosphites and phosphonates, cycloaliphatic phosphites and phosphonates, arsines, lower alcohols, lower ketones, hops, hops acids, aryl pyrazoles, oxazolines, isocyanurates, biguanides, extracts of krameria, hydantoins, pyrollidones, pyrollidone carboxylic acids, pyrollidone carboxylic acid esters, nitrophenols, N-substituted aspartic acids and pyrethroids. Compounds of these classes that have unpleasant odours are referred to herein as malodour compounds.

The aforementioned functional materials are known to include and/or have applied thereto or in a 3-space proximate thereto, fragrance materials that are intended to provide pleasant fragrances which mask the malodour. In addition, the aforementioned defined 3-spaces are known to have introduced therein fragrance materials that are intended to provide pleasant fragrances which mask the malodour present therein and/or introduced thereto. The masking effect is provided by one of two mechanisms. In the first mechanism, the masking fragrance blends with the malodour compound or compounds in an effort to provide a different and more desirable aroma. In the second mechanism the masking fragrance is employed in a large quantity to overwhelm the compound or composition responsible for the malodour.

Both types of mechanisms have serious disadvantages. Neither perfume completely eliminates the perception of malodour and, accordingly, there is a tendency to use increasing quantities and concentrations of fragrance in an effort to eliminate the perception of malodour. Furthermore, the masking effect is an additive effect and so the total odor level in the malodour-masked functional product or in the malodour-masked defined 3-space is increased by consumption of the perfume. Even though the perfume so used may be very pleasant at low concentration, the total odour level in the 3-space proximate the in-use functional product and the total odour level in the aforementioned defined 3-space at the relatively high concentrations required to achieve even moderate masking of the malodour will itself be offensive to the human sense of smell.

Attempts have been made to overcome the aforementioned disadvantages using cyclohexyl-1-ethyl ester substances.

Schleppnik, U.S. Pat. No. 4,622,221, the specification of which is herein incorporated by reference, discloses a method of counteracting a malodour in air caused by a compound selected from the group consisting of lower carboxylic acids, thiols, thiophenols, phenols, lower amines, phosphines and arsines. The method of U.S. Pat. No. 4,622,221 comprises introducing into the air an effective malodour-counteracting amount of cyclohexyl-1-ethyl-n-butyrate or cyclohexyl-1-ethyl acetate whereby the perceived total odor intensity in the air is reduced and the perceived malodour intensity in the air is substantially eliminated. The Schleppnik patent does not, however, disclose or suggest the synergistically-effective cyclohexylethan-1-yl ester mixtures of our invention and their unexpected, unobvious and advantageous properties and use as malodour counteractants as measured physiologically and psychometrically.

O'Connor, U.S. Pat. No. 6,432,891, the specification of which is herein incorporated by reference, discloses a method of counteracting a malodour in a solid or liquid soap or detergent caused by a compound selected from the group consisting of lower carboxylic acids, thiols, thiophenols, lower amines, phosphines, arsines, lower alcohols, and lower ketones. The method disclosed by U.S. Pat. No. 6,432,891 comprises introducing into the solid or liquid soap or detergent an effective malodour-counteracting amount of a malodour-counteracting compound selected from the group consisting of 1-cyclohexyl-ethyl-butyrate, 1-cyclohexyl-ethyl-acetate, 1-cyclohexyl-ethanol, 4-isopropyl-cyclohexyl-propionate, and phenoxyacetic acid 2-hydroxy-ethyl ester. U.S. Pat. No. 6,432,891 further discloses that the perceived total odour intensity in the solid or liquid soap or detergent is reduced, and the perceived malodour intensity in the solid or liquid soap or detergent is substantially eliminated. U.S. Pat. No. 6,432,891 includes examples of the use of mixtures of (i) 1-cyclohexyl-ethyl-acetate and 4-isopropyl-cyclohexyl-propionate in weight rations of 20:80, 50:50 and 80:20 and (ii) 1-cyclohexyl-ethyl-acetate, 1-cyclohexyl-ethanol and 4-isopropyl-cyclohexyl-propionate in attempts to counteract malodours in perfumed shower creams.

The O'Connor patent does not, however, disclose or suggest the synergistically-effective cyclohexylethan-1-yl ester mixtures of our invention and their unexpected, unobvious and advantageous properties and use as malodour counteractants as measured physiologically and psychometrically.

In addition, prior sales have been effected for use as perfumed malodour counteracting compositions of mixtures of 1-cyclohexyl-ethyl-butyrate, 1-cyclohexyl-ethyl-acetate, 1-cyclohexyl-ethanol and 4-isopropyl-cyclohexyl-propionate. Such prior-sold compositions, however, are different in kind from the synergistically-effective cyclohexylethan-1-yl ester mixtures of our invention which have unexpected, unobvious and advantageous properties and use as malodour counteractants as measured physiologically and psychometrically.

### THE INVENTION

Our invention is directed to a synergistically-effective ester mixture consisting essentially of 1-cyclohexylethan-1-yl butyrate having the structure:

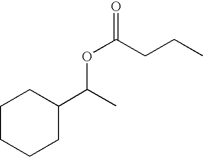

and 1-cyclohexylethan-1-yl acetate having the structure:

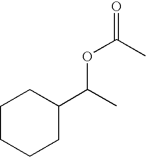

the weight ratio of 1-cyclohexylethan-1-yl butyrate:1-cyclohexylethan-1-yl acetate being from about 20:80 up to about 80:20, in the substantial absence of the compounds:

1-cyclohexylethan-1-ol having the structure:

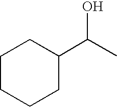

1-(4′-methylethyl)cyclohexylethan-1-yl propionate having the structure:

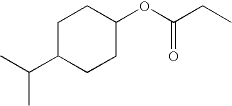

and 2′-hydroxy-1′-ethyl(2-phenoxy)acetate having the structure:

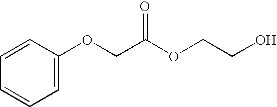

or in the substantial absence of any additional fragrance substances or other malodour counteractant substances. The term, “substantial absence” of a substance is herein intended herein to mean “less than about 1% by weight” of the substance.

Preferably, our invention is directed to a synergistically-effective ester mixture consisting essentially of 1-cyclohexylethan-1-yl butyrate having the structure:

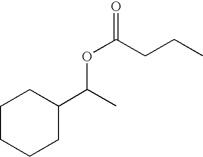

and 1-cyclohexylethan-1-yl acetate having the structure:

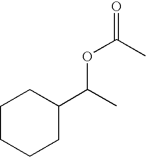

the weight ratio of 1-cyclohexylethan-1-yl butyrate: 1-cyclohexylethan-1-yl acetate being from about 50:50 up to about 80:20, in the substantial absence of the compounds:

1-cyclohexylethan-1-ol having the structure:

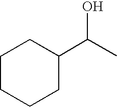

1-(4′-methylethyl)cyclohexylethan-1-yl propionate having the structure:

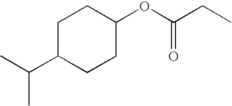

and 2′-hydroxy-1′-ethyl(2-phenoxy)acetate having the structure:

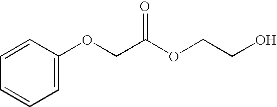

or in the substantial absence of any additional fragrance substances or other malodour counteractant substances.

Our invention is also directed to processes for effectively counteracting and substantially eliminating malodours using such synergistically-effective ester compositions, including:

(a) A process for counteracting a malodour emanating from a solid or liquid malodourous source into a 3-space proximate said source comprising the step of introducing into the 3-space proximate said source a synergistically-effective malodour-counteracting quantity and concentration of one of the aforementioned synergistically-effective ester compositions as a single dose, as a continuous dose over a malodour-counteracting period of time, or as periodic doses over a malodour-counteracting period of time whereby the perceived total malodour intensity is substantially reduced or eliminated; and

(b) A process for counteracting a malodour present in a defined air 3-space comprising the step of introducing into said defined air 3-space a synergistically-effective malodour counteracting quantity and concentration of one of the aforementioned synergistically-effective ester compositions as a single dose, as a continuous dose over a malodour-counteracting period of time, or as periodic doses over a malodour-counteracting period of time whereby the perceived total malodour intensity is substantially reduced or eliminated.

When the 3-space contains, in addition to the malodour, a desirable fragrance, the addition of an effective malodour-counteracting quantity and concentration of the synergistically-effective ester mixture of our invention as a single dose, as a continuous dose over a malodour-counteracting period of time, or as periodic doses over a malodour-counteracting period of time to the 3-space does not eliminate or substantially reduce the perceived odour intensity of the desired fragrance.

The processes of our invention thus effect malodour coverage of a wide variety of solid, liquid and gaseous functional materials including body deodorants, antiperspirants, anti-perspirant-deodorant devices, air fresheners including air freshening devices and solid and liquid air freshening compositions further including room freshener compositions, room deodorants, herbicides, antiviral compositions, fungicides, bactericides, parasiticides, insecticides, depilatory compositions, bleach compositions, bard surface-cleaning compositions, skin cleansing compositions, antimicrobial nail preparations including anti-fungal nail lacquers, hair setting compositions, hair conditioning compositions, trichological lotions, skin lightening compositions, detergent compositions, soap compositions, sunscreen compositions, fabric stain removal compositions, fabric conditioning compositions, fabric anti-wrinkle compositions, steam ironing aroma compositions including stress relief compositions, candle compositions, plant growth regulating compositions, plant growth stimulating compositions, fertilizer compositions, insect attractant compositions, insect repelling compositions, drain cleaning compositions and molluskicide compositions. Such functional materials although useful for their respective purposes, on use thereof emanate odours which are offensive to the human sense of smell.

In addition, the processes of our invention effect malodour coverage in various defined air 3-spaces which have continuously prevailing malodours of constant or variable perception which are offensive to the human sense of smell, for example, indoor gymnasiums, indoor sporting event arenas such as basketball arenas, locker rooms, abattoirs, indoor sporting courts such as handball courts, hair salons, nail salons, tanning salons, beauty salons, tattoo parlors, pig pens, chicken coops, cow barn enclosures, indoor fresh fish markets, fishing boat interiors, horse barn enclosures, rooms containing in-use animal litter containers, kitchens, restaurants, processed food preparation spaces, clothing dry cleaning rooms, garment laundry interiors, cattle cars, plant processing factory rooms, domestic pet shops, zoo animal pens, morgues, autopsy rooms, lavatories, medical patient care rooms, hospital wards and dental patient care rooms.

The aforementioned malodours are caused by chemical compounds of the following classes: aliphatic halohydrins, aliphatic amines, aliphatic N-oxides, dialkylamines, cycloaliphatic amines, cycloaliphatic N-oxides, cyclo-olefinic amines, cyclo-olefinic N-oxides, cycloaromatic amines, cycloaromatic N-oxides, hydroxyalkylamines, imine compounds, amide compounds, amino acids, polypeptides, modified antimicrobial proteins, diureides, nitriles, aliphatic mercaptans, cycloaliphatic mercaptans, mercaptoalkanoic acids, mercaptoalkanoic acid esters, aliphatic monosulfides, disulfides, trisulfides, sulfur oxides, sulfones and sultones, cycloaliphatic monosulfides, disulfides, trisulfides, sulfur oxides, sulfones and sultones, cyclo-olefinic monosulfides, disulfides, trisulfides, sulfur oxides, sulfones and sultones, cycloaromatic monosulfides, disulfides, trisulfides, sulfur oxides, sulfones and sultones, alkali metal bisulfites, sulfites and metabisulfites, isothiocyanates, thiocyanates, dithiocyanates, isothiazolones, isothiazolinones, thiodiazinethiones, halosulfamates, aryl sulfonamides, lower aliphatic carboxylic acids, phenols, phosphines, aliphatic phosphites and phosphonates, cycloaliphatic phosphites and phosphonates, arsines, lower alcohols, lower ketones, hops, hops acids, aryl pyrazoles, oxazolines, isocyanurates, biguanides, extracts of krameria, hydantoins, pyrollidones, pyrollidone carboxylic acids, pyrollidone carboxylic acid esters, nitrophenols, N-substituted aspartic acids and pyrethroids.

The synergistically-effective ester compositions of our invention substantially eliminate the perception of such malodours, while simultaneously refraining from reduction of the perception of pleasant fragrance aromas emanating from the same source or from the proximity of said source.

Examples of functional products which are compositions, effective specific malodourous ingredients or classes of ingredients contained in and/or emanated from said functional products and exemplary defined air 3-spaces where such functional products are used, together with U.S. Patent references setting forth specific examples of the utilities of such functional products are set forth in the following Table I:

TABLE I

| | U.S. | | |
|---|---|---|---|
| Functional | Patent | Ingredient or | Defined Air |
| Product | Reference | Ingredient Class | 3-Space |
| clothing stain | 6,495,510 | diethyl-n-dodecyl | clothing dry |
| removal | | amine oxide | cleaning |
| composition | | | rooms |
| bleach | 6,498,133 | acetonitrile | clothing |
| composition | | | garment |
| | | | laundry |
| | | | interior |
| Skin | 6,497,860 | sodium bisulfite | beauty |
| lightening | | | salon |
| composition | | | |
| herbicide | 6,495,492 | 4-chloro-3-(4- | plant |
| | | cyano-2-fluoro-5- | greenhouse |
| | | phenoxyphenyl)-1- | |
| | | methyl-5- | |
| | | trifluoromethyl- | |
| | | 1H-pyrazole | |
| viruside | 6,468,521 | silver complex of | hospital |
| | | trihydroxy- | ward |
| | | methylaminomethane | |
| fungicide | 6,495,575 | synergistically | plant |
| | | effective amount | greenhouse |
| | | of valinamide and | |
| | | fluazinam | |
| bactericide | 6,207,274; | 4-chloro-5-methyl- | kitchen |
| | 6,475,976 | 2,3-dithiolane; | |
| | | polyhexamethylene- | |
| | | 4-biguanide | |
| parasiticide | 6,265,350 | 1-methylthio | pet shop |
| | | (ethylideneamino)- | |
| | | N-methyl-N- | |
| | | (morpholinothio) | |
| | | carbamate | |
| insecticide | 4,171,340 | 0,0-dimethyl S-(1, | processed |
| | | 2- | food |
| | | dicarboethoxy- | preparation |
| | | ethyl)-dithiophos- | room |
| | | phate | |
| depilitory | 6,479,043 | calcium | hair salon |
| preparation | | thioglycolate | |
| hard surface | 6,440,925 | dodecyldimeth- | kitchen |
| cleaning | | ylamine oxide | |
| preparation | | | |
| skin | 6,491,933 | sodium cocoyl | beauty salon |
| cleansing | | isethionate | |
| preparation | | | |
| anti-fungal | 6,495,124 | macrocyclic | nail salon |
| nail lacquer | | keto-lactones | |
| composition | | | |
| body | 6,325,565 | L-lysine | locker room |
| deodorant | | hexadecylamide | |
| compositon | | | |
| anti- | 6,325,565 | zirconyl | locker room |
| perspirant | | hydroxychloride- | |
| composition | | glycine | |
| | | complex | |
| steam ironing | 6,495,172 | witch hazel | laundry room |
| aroma | | | interior |
| composition | | | |
| antimicrobial | 6,479,039; | N,N- | nail salon |
| nail | 6,495,124 . . . | diethylammonium | |
| preparation | | chloride plus | |
| | | phenol or | |
| | | biguanide; | |
| | | econazole or 1-[2- | |
| | | [(4-chlorophenyl)- | |
| | | methoxy]-2-(2,4- | |
| | | dichlorophenyl) | |
| | | ethyl]-1H- | |
| | | imidazole | |
| hair setting | 6,479,042 | thioglycolic acid | hair salon |
| composition | | | |
| hair | 6,491,902 | polyvinyl amines | hair salon |
| conditioning | | | |
| composition | | | |
| trichological | 6,479,059 | cysteine | hair salon |
| lotion | | | |
| detergent | 6,491,728 | C₁₄-alkylethoxy- | lavatory |
| | | sulfonic acid | |
| soap | 6,479,456 | 3,4,4′-trichloro- | lavatory |
| | | carbanilide | |
| sunscreen | 6,485,713 | p-hydroxydi- | tanning |
| composition | | phenylsulfonate | salon |
| fabric | 6,482,787; | cyclic amine-based | clothing dry |
| conditioning | 6,491,840 | polymers; N,N″- | cleaning |
| compositions | | dioleoyldi- | rooms |
| | | ethylenetriamine | |
| | | and | |
| | | 2,4,6-trimethyl | |
| | | pyridine | |
| candle | 6,111,055; | ester-terminated | restaurant |
| compositions | 5,879,694 | dimer acid-based | |
| | | polyamide; | |
| | | oxidized | |
| | | hydrocarbon oil | |
| plant growth- | 6,444,614 | N-(1,2-dicarboxy- | plant |
| regulating | | ethyl)aspartic | greenhouse |
| composition | | acid | |
| plant growth | 6,444,614 | N,N′-1,2-ethane- | plant |
| stimulating | | diylbis- | greenhouse |
| composition | | aspartic acid | |
| fertilizer | 6,500,222 | diureides | plant |
| composition | | | greenhouse |
| insect | 5,928,634; | sucrose, fructose, | plant |
| attractant | 6,410,567 | maltose and the | greenhouse |
| | | lithium salt of | |
| | | saccharin; | |
| | | disparlure plus a | |
| | | tetrapyrrole | |
| insect | 6,451,844; | menthyl-2- | kitchen |
| repellent | 6,306,415 | pyrollidone-5- | |
| | | carboxylate; 1:1:1 | |
| | | octanoic acid, | |
| | | nonanoic acid | |
| | | and decanoic acid | |
| drain | 6,479,444 | C₁₄₋₁₈alkyl | kitchen |
| cleaning | | sulfobetaine plus | |
| composition | | NaOCl | |
| room | 4,622,221 | d-limonene | kitchen |
| freshener | | | |
| composition | | | |
| malodourous | 6,491,962 | amino acids plus | plant |
| plant | | sodium hypochlorite | processing |
| processing | | | factory room |
| environment | | | |
| molluskicide | 6,265,350 | 1,3-oxazolines and | fishing boat |
| | | 1,3-thiazolines | interior |

The specification of each of the patents a listed in Table I is herein incorporated by reference as if set forth in their entirety.

When used in conjunction with malodourous solid or liquid functional products the synergistically effective ester malodour counteracting mixtures of our invention may comprise from about 0.01% by weight up to about 15.0% by weight of the solid or liquid functional product as set forth and as specifically exemplified in Example 1, preferably from about 0.025% up to about 10% and more preferably from about 0.05% up to about 4.0%.

When used in conjunction with malodourous gaseous functional products, the synergistically effective ester malodour-counteracting mixtures of our invention may comprise from about 0.01 up to about 1 mg per cubic meter of the air 3-space which contains the malodour to be counteracted.

Specific details concerning the functional products, soaps and detergents, are set forth in U.S. Pat. No. 6,432,891, the specification of which is herein incorporated by reference. Specific details concerning the air freshener composition functional product are set forth in U.S. Pat. No. 4,622,221, the specification of which is herein incorporated by reference.

Examples of functional products which are air freshener devices used in the practice of our invention together with U.S. Patent references setting forth specific examples of the utilities of such air freshener devices are set forth in the following Table II:

TABLE II

| Air Freshening | U.S. Patent |
|---|---|
| Device | Reference |
| Night Light | 6,478,440 |
| Air Freshener | |
| Light Bulb | 4,184,099 |
| Room Freshener | 4,837,421 |

The specification of each of the patents listed in Table II is herein incorporated by reference.

An example of a body deodorant/anti-perspirant device used in the practice of our invention is that shown in FIG. 7 of U.S. Pat. No. 6,325,565 and described in detail in said U.S. Pat. No. 6,325,565 the specification of which is incorporated herein by reference.

The synergistically effective ester composition of our invention may be delivered to the location of its use by means known to those having ordinary skill in the art, including (i) directing to the desired site the ester composition in the form of a gas phase vapor, (ii) spraying the ester composition of our invention as an aerosol continuously, or as pulsed doses, or as an initial dose, (iii) forming microcapsules containing the ester composition surrounded by a wall of, for example, gelatin formed by a coacervation process and then delivering the microcapsules to the site of use where the ester compositions of our invention are controllably released or (iv) in the case of a solid malodorous porous functional product, injecting into the interstices of the solid functional product as an initial dose, or in continuous doses or in pulsed doses, the ester composition of our invention. When the delivery of the synergistically-effective ester composition to the desired location is carried out by directing a gas phase vapor to the desired site, the apparatus and process disclosed in Young et al., application for U.S. patent Ser. No. 10/212,349 filed on Aug. 5, 2002, the disclosure of which is herein incorporated by reference, may be used. When the delivery of the synergistically-effective ester composition of our invention is carried out by means of introduction of control-release microcapsules to the desired location, microcapsules produced according to the disclosures of U.S. Pat. No. 6,491,902 or U.S. Pat. No. 6,485,736 may be used. The specifications of each of U.S. Pat. Nos. 6,491,902 and 6,485,736 are herein incorporated by reference.

When the delivery of the synergistically-effective ester composition of our invention is carried out in pulses, the time period for each pulse is herein indicated as “pulse duration” and the time between the pulses is herein indicated as “inter-stimulus intervals” or ‘ISI”. The process of our invention wherein the synergistically-effective ester composition is delivered to the desired site in pulses or “periodic doses” over a malodour-counteracting period of time is governed by the model:

Z=Zₒ+A(1−B^{X})+C(1−D^{Y})

wherein Z is the percentage of odour pulses correctly identified;

Y is the pulse duration measured in units of time;

X is the inter-stimulus interval measured in units of time;

Zₒ is a measure, as a scaled value of from −100 up to 100 of the sensitivity of the subjects to the odour detected;

A and B are parameters, each defining the effects of the inter-stimulus interval, X, and stimulus frequency, φ, on the percentage of odour pulses correctly identified, Z, and each is a measure of the degree of adaptation to malodours and habituation as measured by the tangent slope to the X-Z curve at a given point, (X₁, Z₁), Δ F(X₁, Z₁);

C is a concentration parameter and is a measure of the magnitude of the tangent slope to the Y-Z curve at a given point, (Y₁, Z₁), Δ F(Y₁, Z₁); C and D are parameters, each being measures of the effects of pulse duration, Y, and the inter-stimulus interval, X on the percentage of odour pulses correctly identified, Z, and each is a measure of the angle of inclination, θ, of the normal to the tangent plane to the X-Y-Z surface at a point (X₁,Y₁,Z₁) wherein θ=arccos[|ΔF (X₁,Y₁,Z₁)·|k|/{∥ΔF (X₁, Y₁,Z₁)∥}] with |k representing the “z” axis vector and (i) in the case of the weight ratio range of the 1-cyclohexylethan-1-yl butyrate: 1-cyclohexylethan-1-yl acetate being from about 20:80 up to about 80:20, Zₒ being in the range of from about −45 up to about −15; A being in the range of from about 50 up to about 65; B being in the range of from about 0.93 up to about 0.97; C being in the range of from about 68 up to about 71; D being in the range of from about 0.98 up to about 0.99; and with (ii) in the case of the weight ratio range of the 1-cyclohexylethan-1-yl butyrate: 1-cyclohexylethan-1-yl acetate being in the range of from about 50:50 up to about 80:20, Zₒ being in the range of from about −45 up to about −25; A being in the range of from about 55 up to about 65; B being in the range of from about 0.95 up to about 0.97; C being in the range of from about 68 up to about 70; and D being in the range of from about 0.98 up to about 0.99.

A detailed discussion of the model

Z=Zₒ+A(1−B^{X})+C(1−D^{Y})

as it applies to individual malodour molecules and individual fragrance molecules is set forth in Jacob et al., “Psychometric Evaluation of Responses to Pleasant and Malodour Stimulation in Human Subjects; Adaptation, Dose Response and Gender Differences”, Int. J. Psychophysiology, (2003). An additional discussion of the model as it applies to olfaction is set forth in Wang et al., “The correlation between physiological and psychological responses to odor stimulation in human subjects” Clinical Neurophysiology 113 (2002) 542-551 at page 546.

### BRIEF DESCRIPTION OF THE DRAWINGS

FIG. 1 is a schematic diagram of the olfactometer used in carrying out the procedures of Example I.

FIG. 2 is a schematic diagram of the left side of the head of a human mammal indicating placement of electrodes and related brain regions in accordance with “The International 10-20 System of Electrode Placement” as utilized for the procedures and as described in detail in Example I.

FIG. 3 is a schematic diagram of the top view of the head of the human mammal of FIG. 2.

FIG. 4A is a set of bar graphs showing the measurements of the counteraction of a valeric acid malodour by a 20:80 mixture of cyclohexylethan-1-yl butyrate and 1-cyclohexylethan-1-yl acetate as a function of the magnitude of the standardized organoleptic event-related potential, “OERP” in Example I.

FIG. 4B is a set of bar graphs showing the measurements of the counteraction of a valeric acid malodour by a 20:80 mixture of cyclohexylethan-l-yl butyrate and 1-cyclohexylethan-1-yl acetate as a function of % psychometric-cognitive recognition of odor or “% perception” in Example I.

FIG. 5A is a set of bar graphs showing the measurements of the counteraction of a valeric acid malodour by a 50:50 mixture of cyclohexylethan-I-yl butyrate and 1-cyclohexylethan-1-yl acetate as a function of the magnitude of the standardized organoleptic event-related potential, “OERP” in Example I.

FIG. 5B is a set of bar graphs showing the measurements of the counteraction of a valeric acid malodour by a 50:50 mixture of cyclohexylethan-1-yl butyrate and 1-cyclohexylethan-1-yl acetate as a function of % psychometric-cognitive recognition of odor or “% perception” in Example I.

FIG. 6A is a set of bar graphs showing the measurements of the counteraction of a valeric acid malodour by a 80:20 mixture of cyclohexylethan-1-yl butyrate and 1-cyclohexylethan-1-yl acetate as a function of the magnitude of the standardized organoleptic event-related potential, “OERP” in Example I.

FIG. 6B is a set of bar graphs showing the measurements of the counteraction of a valeric acid malodour by a 80:20 mixture of cyclohexylethan-1-yl butyrate and 1-cyclohexylethan-1-yl acetate as a function of % psychometric-cognitive recognition of odor or “% perception” in Example I.

FIG. 7A is a set of bar graphs showing the measurements of the counteraction of an amyl acetate “pleasant aroma” by a 20:80 mixture of cyclohexylethan-1-yl butyrate and 1-cyclohexylethan-1-yl acetate as a function of the magnitude of the standardized organoleptic event-related potential, “OERP” in Example I.

FIG. 7B is a set of bar graphs showing the measurements of the counteraction of an amyl acetate “pleasant aroma” by a 20:80 mixture of cyclohexylethan-1-yl butyrate and 1-cyclohexylethan-1-yl acetate as a function of % psychometric-cognitive recognition of odor or “% perception” in Example I.

FIG. 8A is a set of bar graphs showing the comparison of the % inhibition of OERP for ester mixtures of our invention vs. the individual components of the ester mixtures of our invention as set forth in detail in Example I.

FIG. 8B is a set for bar graphs showing the comparison of the % inhibition of perception forester mixtures of our invention vs. the individual components of the ester mixtures of our invention as set forth in detail in Example I.

FIG. 9A is a dose-stimulus-frequency set of bar graphs for the malodour, n-butyric acid shown in three dimensions.

FIG. 9B is a dose-stimulus-frequency set of bar graphs for the “pleasant” odour, amyl acetate, shown in three dimensions.

FIG. 9C is a set of adaptation curves for the malodour, n-butyric acid with the vertical axis being the “Z” axis for % odor pulses correctly defined and the horizontal axis being the “X” axis for inter-stimulus interval.

FIG. 9D is a set of adaptation curves for the “pleasant” odour, amyl acetate, with the vertical axis being the “X’ axis for % odour pulses correctly defined and the horizontal axis being the “X” axis for inter-stimulus interval.

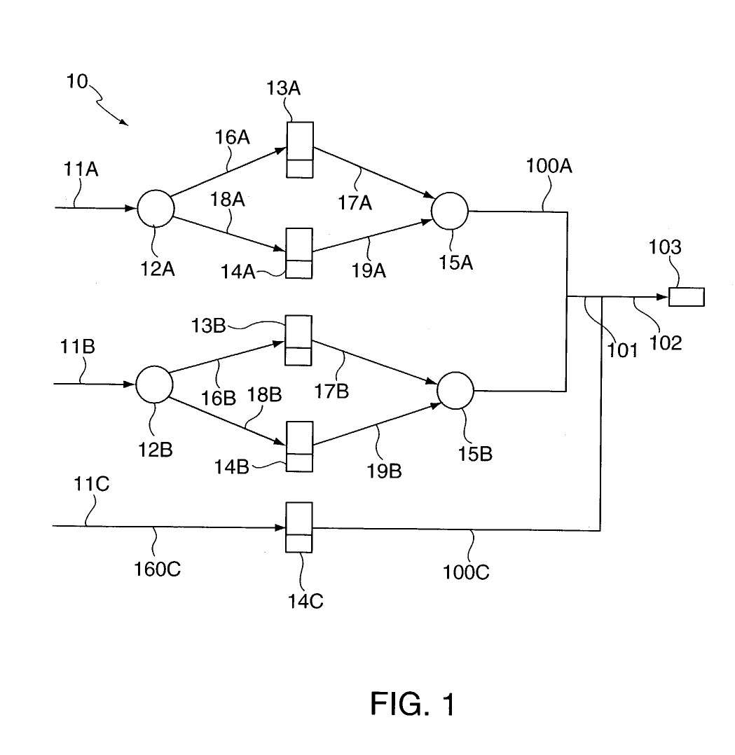

### EXAMPLE I AND DETAILED DESCRIPTION OF FIGS. 1-8B OF THE DRAWINGS

This example is indicative of properties of different proportions of binary combinations of cyclohexylethan-1-yl butyrate and cyclohexylethan-1-yl acetate against the malodour valeric acid.

Mixtures of cyclohexylethan-1-yl butyrate and cyclohexylethan-1-yl acetate, hereinafter referred to as the “ester mixtures” were used as the counteractant and n-valeric acid, hereinafter referred to as “VA” as the malodour. The mixtures were diluted with dipropylene glycol to final concentrations of 0.01% for each ester mixture and 2% for the VA.

Three weight ratio combinations of ester mixture components were tested: cyclohexylethan-1-yl butyrate:cyclohexylethan-1-yl acetate at 20:80, 50:50 and 80:20. For each mixture between 7-12 subjects were used, attempting to balance the genders, giving a total of 152 subjects. The single individual ester components, cyclohexylethan-1-yl butyrate and cyclohexylethan-1-yl acetate were repeated at the concentration, 0.01% for the purposes of comparison.

The ability of the 50:50 mixture of the cyclohexylethan-1-yl butyrate and cyclohexylethan-1-yl acetate was tested against a pleasant odour, amyl acetate, as a control.

Delivery of Odour

As shown in FIG. 1, odours are delivered to the nostril 103 by using an 12A olfactometer system 10. There are four teflon-lined solenoid three-way valves, 12A, 12B, 15A and 15B (Cole Palmer, Bishops Stortford, UK) in the system. The solenoid valves are switched by Darlington drivers under computer control via the digital output of an A/D converter (CED 1401, Cambridge, UK).

Through lines 11A, 11B and 11C, air is pumped by a microprocessor-controlled pump (J. D. Technical Services, Old Ynysybwl, Wales, UK). The airflow is split into three directions and connected in parallel to three flow meters (Platon Flowbits, Basingstoke, UK), set to 1 L/min each. The outputs of the flow meters are diverted to three reservoir destinations, respectively, (i) 13A or 14A, via lines 16A or 18A, respectively, (ii) 13B or 14B via lines 16B or 18B, respectively, and (iii) 14C via line 160C. Line 160C is connected to a glass reservoir 14C containing warm water, and are then joined with the output of other two sets of reservoirs 13A-13B and 14A-14B,. The other two outlets of the flow meters are connected via lines 11A and 11B to valves 12A and 12B, respectively. Air flowing through each of the two sets of two parallel lines 16A-18A, and 16B-18B, leaving each of valves 12A and 12B, respectively, is fed to two glass reservoirs containing (i) either warm water in the case of reservoir 14A or VA or amyl acetate in the case of reservoir 13A or (ii) warm water in the case of reservoir 14B or one of the ester mixtures in the case of reservoir 13B, and then passed via lines 17A and 19A to valve 15A, or via lines 17B and 19B to valve 15B. The airflow leaving valves 15A and 15B is then combined with the airflow emanating from reservoir 14C passing through line 100C from the output of airflow through line 11C and the combined airflow is passed through line 102 into one of the nostrils 103. The nostril 103 is blocked by a self-expanding foam bung with the odour delivery tube passing through its centre. This prevents back-flow and forces the air to pass through the nasopharynx into the oral cavity.

Odor concentration is altered by varying the pulse duration; that is the length of time that the air flow is diverted through odor reservoir 13A and/or 13B directed by means of appropriate adjustment of the solenoid valves 12A and/or 12B. The pulse of the odour vapour being conveyed via lines 100A and 100B is joined in line 102 with the continuous flow air-line 100C diluting it 1:3. Thus, for the 100 msec. pulse, 5 milliliters of air plus odour vapour is delivered, which represents 1.667 milliliters of odour vapour. In the case of reservoir 13A containing amyl acetate, the concentration of amyl acetate emanating from reservoir 13A through line 17A and line 100A is 518 micromoles/liter, or “518 μM” at 35° C. However the vapour temperature decreases to 24° C. in line 101 prior to entry to line 102 where the concentration of the amyl acetate is 270 μM. Subsequent to dilution with air in line 102, the concentration, resulting from 1:3 air dilution is 90 μM. When the air-vapour delivery rate is 3 liters per minute, the delivery rate of the amyl acetate is 270 μmoles/minute or 4.5 μmoles/second. Accordingly, 0.45 μmoles of amyl acetate is delivered in a 100 μsecond pulse.

During each experimental run, the four valves 12A, 12B, 15A and 15B are specially set by computer program. Airflow through the water reservoirs 14A, 14B and 14C is set to be continuous, but the airflow is diverted to pass through the odour reservoirs 13A, containing the VA or amyl acetate and 13B containing the ester mixture when required, for pulses of 100 microseconds duration and 10 second inter-stimulus interval. For each stimulus block 15 odour pulses are delivered.

Each experiment is divided into four sections. Each section is separated by a two-minute break. The four sections and their sequence are as follows:

(1) VA or amyl acetate (2%) alone;

(2) VA or amyl acetate (2%) plus binary ester mixture (20:80, 50:50, 80:20, 0.01% each ester mixture);

(3) VA or amyl acetate (2%) alone;

(4) Ester mixture alone.

Subjects

Subjects are between 18-25 years old from the student population of the University and none has a history of olfactory dysfunction or respiratory disease. The protocol is explained to each subject and informed consent is obtained from each subject. During the experiment each of the subjects is seated in a comfortable chair in a test booth having a controlled environment. Each of the subjects is given a visual stimulus (10-cm TV playing silent cartoons) that maintains alertness (eliminated alpha-wave production) while minimizing eye movements and reducing blinking. Each of the subjects wears headphones through which white noise is played to eliminate auditory cues. Each of the subjects is given a button to press for the psychometric test so the subject could record odour pulse detection.

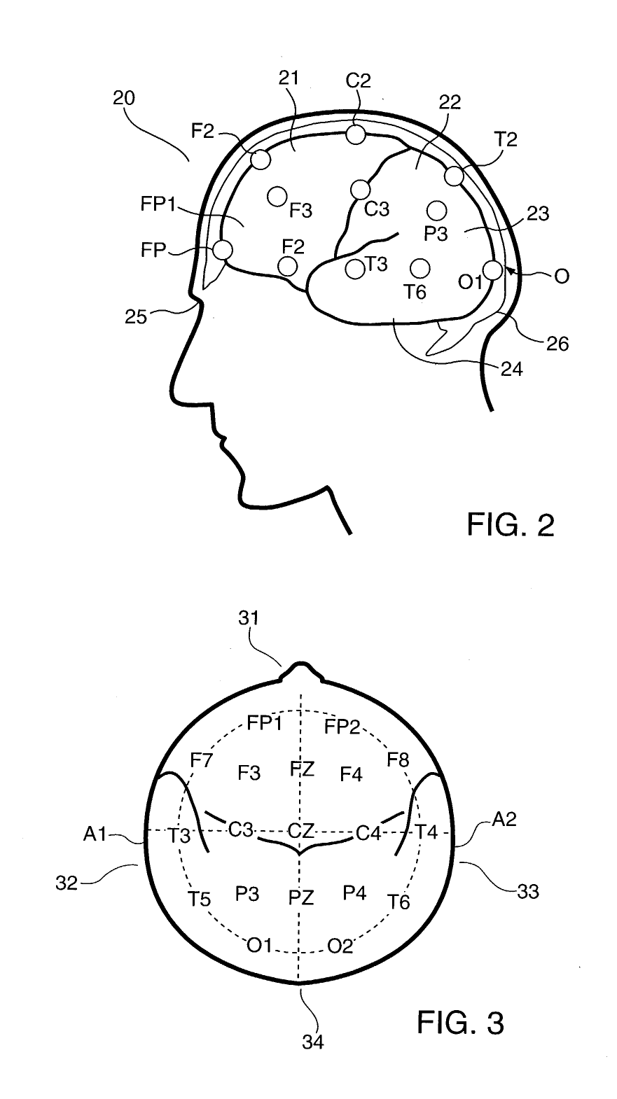

For the purpose of this experiment, electrodes are placed, as set forth in FIG. 2, on the head, indicated by reference numeral 20 in FIG. 2, of each subject in accordance with The International 10-20 System of Electrode Placement as explained in detail in Chapter 9, at pages 100-126, particularly page 110 of the text, James Hasset (1978) “A Primer of Psychophysiology”. The International 10-20 System of Electrode Placement is the most widely used method to describe the location of scalp electrodes. The 10-20 system is based on the relationship between the location of an electrode and the underlying area of cerebral cortex. Each site is assigned a reference letter in order to facilitate identification of the lobe and a reference number or another reference letter to identify the hemisphere location.

As set forth in FIG. 2 and FIG. 3, the reference letters used are: “F”—Frontal lobe, the region of which is indicated by reference numeral 21; “IT”—Temporal lobe, the region of which is indicated by reference numeral 24; “C”—Central lobe, “P”—Parietal lobe, the region of which is indicated by reference numeral 22; “O”—Occipital lobe, the region of which is indicated by reference numeral 23. It is herewith emphasized that there is no central lobe in the cerebral cortex. Accordingly, the reference letter “C” is used herein for identification purposes only. Even numbers affiliated with the reference letters to wit: 2, 4, 6 and 8 refer to the right hemisphere, 33 and odd numbers affiliated with the reference letters in FIG. 2 and FIG. 3, to wit:1, 3, 5, 7 refer to the left hemisphere, 32. The reference letter, “Z” refers to an electrode placed on the midline. The smaller the number, the closer the position to the midline. The reference,“FP” stands for “Front polar”, the region of which is indicated by the references FP1 and FP2; “Nasion” is the point between the forehead and nose, indicated by reference numeral 25. “Inion” is the bump at the back portion, 34 of the skull, indicated by reference numeral 26. The numerals “10 ” and “20 ” of the term “10-20 system” refer to the 10% and 20% interelectrode distance. Thus, as shown in FIG. 2 electrodes are placed at the following reference locations at the left side A1 of each of the subjects, indicated by reference numeral 32: FP1, F3, C3, T3, P3 and O1. Correspondingly, electrodes are placed at the following reference locations at the right side A2 of each of the subjects, indicated by reference numeral 33: FP2, F2, C2, T2 and T6.

Recording and Analysis of Olfactory Event-Related Potentials

Olfactory event-related potentials also referred to herein as OERP's are recorded using electroencephalography (EEG-5200, Nihon Kohden UK, Middlesex, UK). Electrodes are placed as set forth diagrammatically in FIG. 2, with locations specified according to FIG. 3 according to the international 10/20 system, with reference to the side indicated by reference A1 and reference numeral 32 and an earth electrode is placed on the forehead of each subject proximate the location indicated by reference numeral 31 in FIG. 3, in the region of the location indicated by reference FP1. Data transmitted via electrode placed on each subject at reference numeral CZ are recorded and analysed in this study. Traces contaminated with eye movement artifacts detected via electrode located on each subject at reference numeral FP1 are discarded. The time constant of the amplifiers is set to 0.5 seconds (≈1 Hz high pass filter) and the data are low pass filtered at 35 Hz. An additional 50 Hz notch filter is used.

Analogue data were sent to a laboratory interface (CED1401), digitized at 100 Hz and analyzed following signal averaging on a computer using “SIGNAL” analysis software (CED, Cambridge, UK). The “SIGNAL” analysis software and relevant use thereof is referred to at paragraphs 2.5 and 2.6 in Wang et al., “The correlation between physiological and psychological responses to odor stimulation in human subjects”Clinical Neurophysiology 113 (2002) 542-551.

The OERPs are measured from digitized records using the aforementioned SIGNAL analysis software (CED, Cambridge). In conformity with other studies of organoleptic event-related potentials, the OERP is taken to be the N1-P₂/P₃ waveform. The peak value or “amplitude” of the OERP is measured between manually set cursors. These are set just before N₁ and just after the N₁-P₂/P₃ waveform. Amplitude measurement could be semi-automated, removing subjectivity from the process. The noise, measured from the pre-stimulus baseline, was subtracted from this value.

Psychometric Tests

The psychometric tests are performed during the same experiment as the EEG recording. Subjects are presented with VA alone, VA in combination with ester mixture or ester mixture alone using the protocol described. Each of the subjects is requested to record the number of odour pulses which is detected by pressing a button connected to a laboratory interface. The timing and number of pulses is stored on the computer hard drive along with the EEG traces.

Statistical Analysis

Statistical differences of the data were evaluated by Student's t-test and considered significant at P<0.05. Data were expressed as mean±standard error of the mean (n=number of observation).

Results

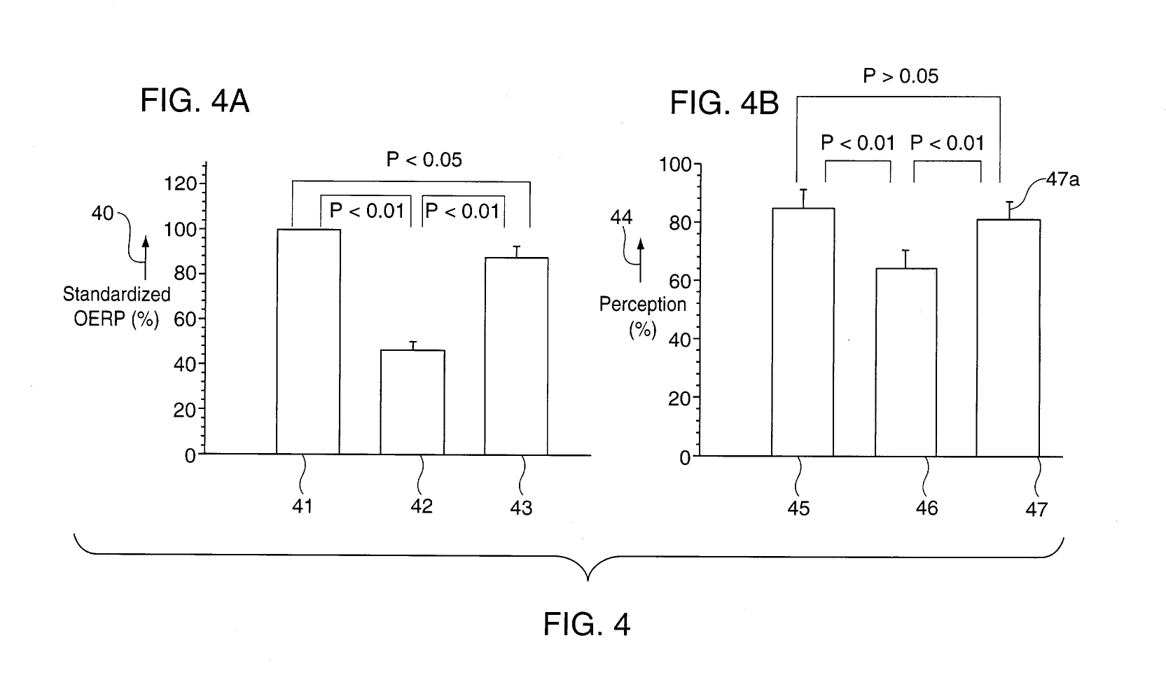

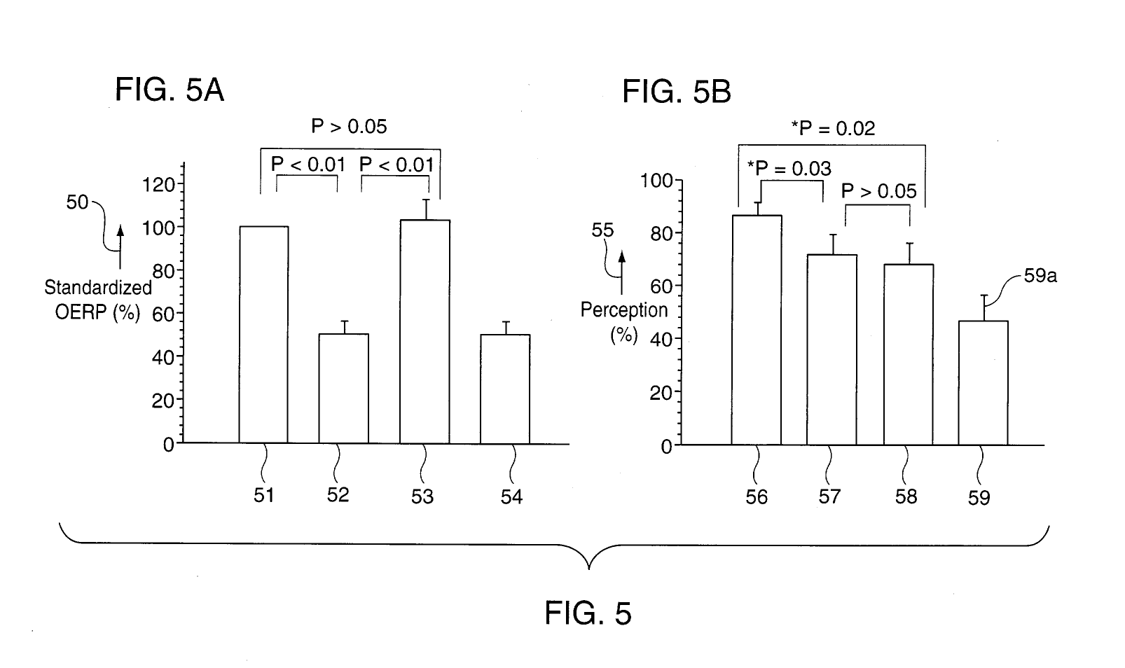

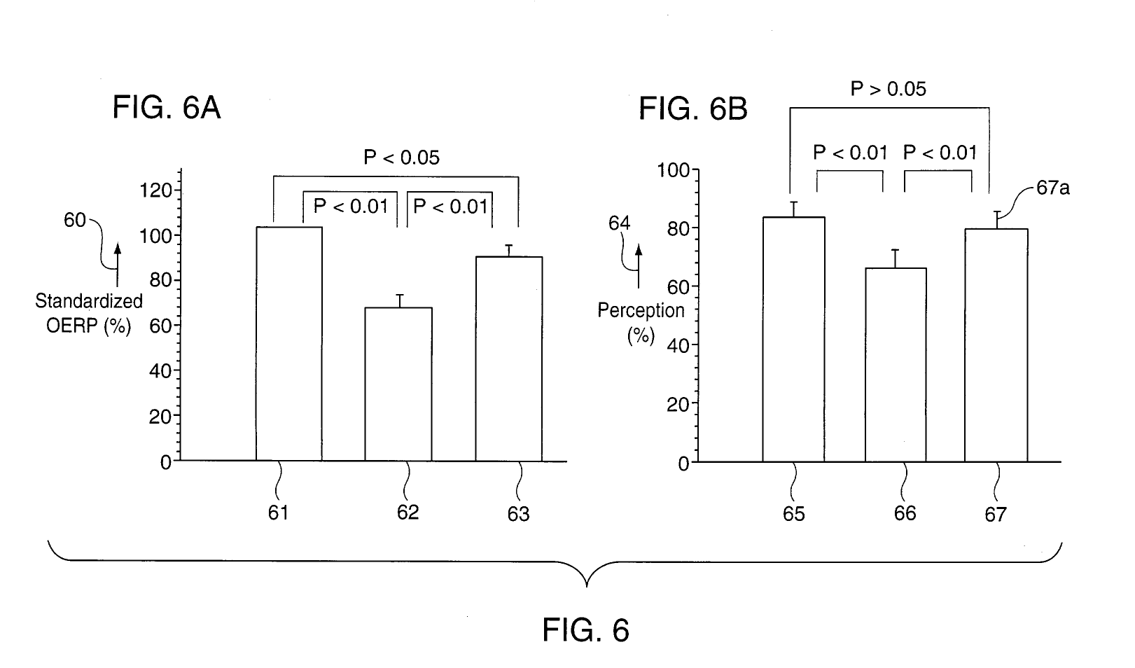

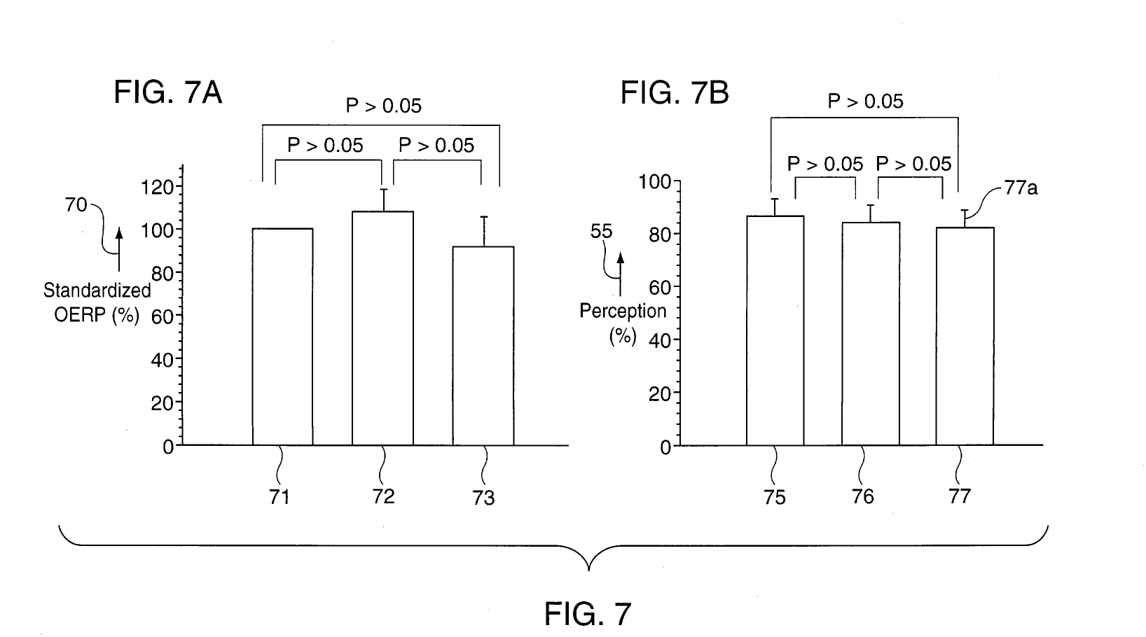

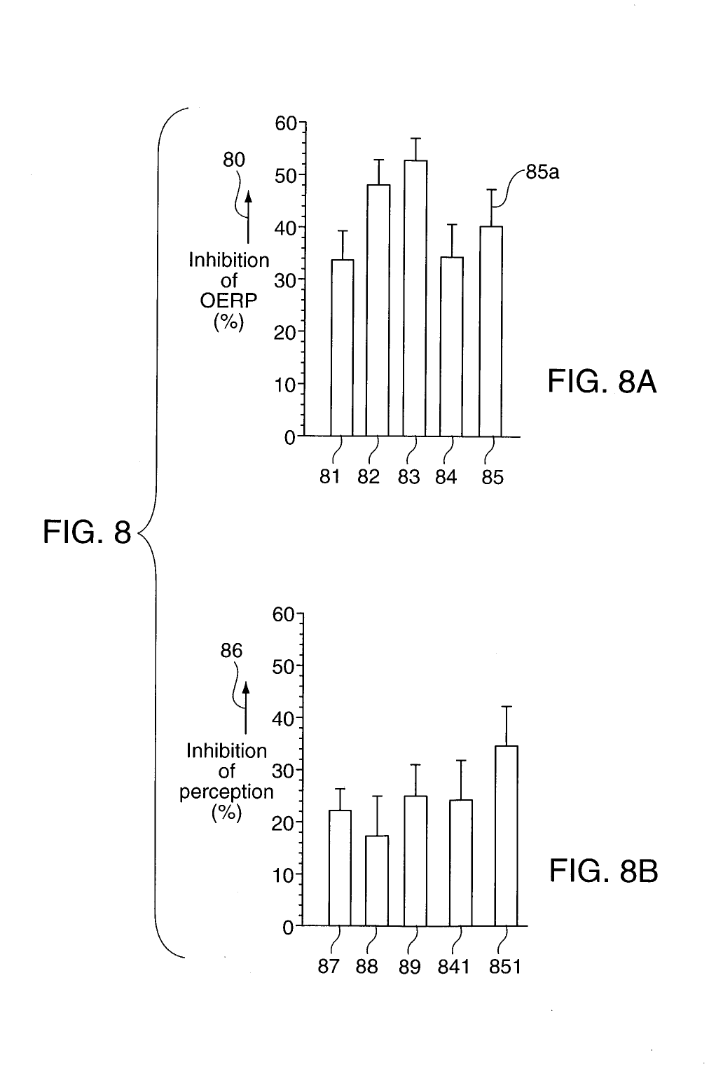

Referring to FIG. 4A, FIG. 5A, FIG. 6A, FIG. 7A, and FIG. 8A the “Y” axis, represented by reference numerals 40, 50, 60, 70 and 80 provides the magnitude of the standardized organoleptic event related potential, “OERP” in each of FIGS. 5A, 6A, 7A and 8A, respectively. In the data represented by FIGS. 4A, 5A and 6A, n=10. In the data represented by FIG. 7A, n=7.

In FIG. 4A, showing the measurements of the counteraction of a valeric acid malodour by a 20:80 mixture of cyclohexylethan-1-yl butyrate and 1-cyclohexylethan-1-yl acetate, the bar graphs indicated by reference numerals 41 and 43 are for 2% n-valeric acid also termed herein,“VA”. The bar graph indicated by reference numeral 42 is for a mixture of “VA” at a level of 2% and a 20:80 mixture of 1-cyclohexylethan-1-yl butyrate: 1-cyclohexylethan-1-yl acetate at levels of 0.004% and 0.016%, respectively.

In FIG. 5A, showing the measurements of the counteraction of a valeric acid malodour by a 50:50 mixture of cyclohexylethan-1-yl butyrate and 1-cyclohexylethan-1-yl acetate, the bar graphs indicated by reference numerals 51 and 53 are for 2% n-valeric acid also termed herein,“VA”. The bar graph indicated by reference numeral 52 is for a mixture of “VA”;at a level of 2% and a 50:50 mixture of 1-cyclohcxylethan-1-yl butyrate: 1-cyclohexylethan-1-yl acetate at levels of 0.01% and 0.01%, respectively. The bar graph indicated by reference numeral 54 is for a 50:50 mixture of 1-cyclohexylethan-1-yl butyrate:1-cyclohexylethan-1-yl acetate at levels of 0.01% and 0.01%, respectively.

In FIG. 6A, showing the measurements of the counteraction of a valeric acid malodour by an 80:20 mixture of cyclohexylethan-1-yl butyrate and 1-cyclohexylethan-1-yl acetate, the bar graphs indicated by reference numerals 61 and 63 are for 2% n-valeric acid also termed herein,“VA”. The bar graph indicated by reference numeral 62 is for a mixture of “VA” at a level of 2% and an 80:20 mixture of 1-cyclohexylethan-1-yl butyrate: 1-cyclohexylethan-1-yl acetate at levels of 0.016% and 0.004%, respectively.

In FIG. 7A, showing the measurements of the counteraction of an amyl acetate pleasant aroma by a 20:80 mixture of cyclohexylethan-1-yl butyrate and 1-cyclohexylethan-1-yl acetate, the bar graphs indicated by reference numerals 71 and 73 are for 2% amyl acetate. The bar graph indicated by reference numeral 72 is for a mixture of amyl acetate at a level of 2% and a 20:80 mixture of 1-cyclohexylethan-1-yl butyrate: 1-cyclohexylethan-1-yl acetate at levels of 0.004% and 0.016%, respectively.

In FIG. 8A, showing the comparison of the % inhibition of OERP for ester mixtures of our invention vs. the individual components of the ester mixtures of our invention, the bar graph indicated by reference numeral 81 is for an 80:20 mixture of 1-cyclohexylethan-1-yl butyrate: 1-cyclohexylethan-1-yl acetate; the bar graph indicated by reference numeral 82 is for a 50:50 mixture of 1-cyclohexylethan-1-yl butyrate: 1-cyclohexylethan-1-yl acetate; the bar graph indicated by reference numeral 83 is for a 20:80 mixture of 1-cyclohexylethan-1-yl butyrate:1-cyclohexylethan-1-yl acetate; the bar graph indicated by reference numeral 84 is for 1-cyclohexylethan-1-yl butyrate, alone; and the bar graph indicated by reference numeral 85 is for 1-cyclohexylethan-1-yl acetate, alone. Reference numeral 85a indicates the standard deviation for the bar graph indicated by reference numeral 85.

Referring to FIG. 4B, FIG. 5B, FIG. 6B and FIG. 7B the “Y” axis, represented by reference numerals 44, 55, 64 and 74 provides the magnitude of the % psychometric-cognitive recognition of odour or malodour as the case may be, also referred to as “% perception” in each of FIGS. 5B, 6B and 7B respectively. In the data represented by FIGS. 4B, 5B and 6B, n=10. In the data represented by FIG. 7B, n=7. In FIG. 8B, the “Y” axis, represented by reference numeral 86 provides the magnitude of the % inhibition of perception.

In FIG. 4B, showing the measurements of the counteraction of a valeric acid malodour by a 20:80 mixture of cyclohexylethan-1-yl butyrate and 1-cyclohexylethan-1-yl acetate, the bar graphs indicated by reference numerals 45 and 47 are for 2% n-valeric acid also termed herein,“VA”. The bar graph indicated by reference numeral 46 is for a mixture of “VA” at a level of 2% and a 20:80 mixture of 1-cyclohexylethan-1-yl butyrate: 1-cyclohexylethan-1-yl acetate at levels of 0.004% and 0.016%, respectively. Reference numeral 47a indicates the standard deviation for the bar graph indicated by reference numeral 47.

In FIG. 5B, showing the measurements of the counteraction of a valeric acid malodour by a 50:50 mixture of cyclohexylethan-1-yl butyrate and 1-cyclohexylethan-1-yl acetate, the bar graphs indicated by reference numerals 56 and 58 are for 2% n-valeric acid also termed herein,“VA”. The bar graph indicated by reference numeral 57 is for a mixture of “VA” at a level of 2% and a 50:50 mixture of 1-cyclohexylethan-1-yl butyrate: 1-cyclohexylethan-1-yl acetate at levels of 0.01% and 0.01% , respectively. The bar graph indicated by reference numeral 59 is for a 50:50 mixture of 1-cyclohexylethan-1-yl butyrate: 1-cyclohexylethan-1-yl acetate at levels of 0.01% and 0.01% , respectively. Reference numeral 59 a indicates the standard deviation for the bar graph indicated by reference numeral 59.

In FIG. 6B, showing the measurements of the counteraction of a valeric acid malodour by an 80:20 mixture of cyclohexylethan-1-yl butyrate and 1-cyclohexylethan-1-yl acetate, the bar graphs indicated by reference numerals 65 and 67 are for 2% n-valeric acid also termed herein,“VA”. The bar graph indicated by reference numeral 66 is for a mixture of “VA” at a level of 2% and an 80:20 mixture of 1-cyclohexylethan-1-yl butyrate:1-cyclohexylethan-1-yl acetate at levels of 0.016% and 0.004%, respectively. Reference numeral 67a indicates the standard deviation for the bar graph indicated by reference numeral 67.

In FIG. 7B, showing the measurements of the counteraction of an amyl acetate pleasant aroma by a 20:80 mixture of cyclohexylethan-1-yl butyrate and 1-cyclohexylethan-1-yl acetate, the bar graphs indicated by reference numerals 75 and 77 are for 2% amyl acetate. The bar graph indicated by reference numeral 72 is for a mixture of amyl acetate at a level of 2% and a 20:80 mixture of 1-cyclohexylethan-1-yl butyrate: 1-cyclohexylethan-1-yl acetate at levels of 0.004% and 0.016%, respectively. Reference numeral 77a indicates the standard deviation for the bar graph indicated by reference numeral 77.

In FIG. 8B, showing the comparison of the % inhibition of perception for ester mixtures of our invention vs. the individual components of the ester mixtures of our invention, the bar graph indicated by reference numeral 87 is for an 80:20 mixture of 1-cyclohexylethan-1-yl butyrate: 1-cyclohexylethan-1-yl acetate; the bar graph indicated by reference numeral 88 is for a 50:50 mixture of 1-cyclohexylethan-1-yl butyrate: 1-cyclohexylethan-1-yl acetate; the bar graph indicated by reference numeral 89 is for a 20:80 mixture of 1-cyclohexylethan-1-yl butyrate: 1-cyclohexylethan-1-yl acetate; the bar graph indicated by reference numeral 841 is for 1-cyclohexylethan-1-yl butyrate, alone; and the bar graph indicated by reference numeral 851 is for 1-cyclohexylethan-1-yl acetate, alone.

Conclusion

As a result of the ester mixtures of our invention being evaluated in the proportions; 20:80, 50:50 and 80:20 as set forth in FIGS. 4-8, inclusive, whereby the results of the physiological and psychometric tests were compared the following conclusions are herewith made:

(a) The ester mixtures of our invention in the ratios of 20:80, 50:50 and 80:20 did not counteract a control odour as confirmed by the results summarized in FIG. 7A and FIG. 7B.

(b) All combinations of the ester mixtures of our invention caused significant counteraction of valeric acid in the ratios of 20:80, 50:50 and 80:20 in both physiological and psychometric testing as confirmed by the results summarized in FIGS. 4A, 4B, 5A, 5B, 6A, 6B, 8A and 8B.

(c) Performing an independent samples two-tailed t-test on the data demonstrated that:

(i) The 20:80 weight ratio 1-cyclohexylethan-1-yl butyrate: 1-cyclohexylethan-1-yl acetate mixture performed unexpectedly and significantly better than either 1-cyclohexylethan-1-yl butyrate or 1-cyclohexylethan-1-yl acetate alone (p=0.027 and 0.022, respectively) on the basis of the physiological test; and

(ii) The 50:50 weight ratio1-cyclohexylethan-1-yl butyrate: 1-cyclohexylethan-1-yl acetate mixture performed better than the 1-cyclohexylethan-1-yl butyrate and the 1-cyclohexylethan-1-yl acetate alone on the basis of the physiological test (p=0.099 and 0.49, respectively).

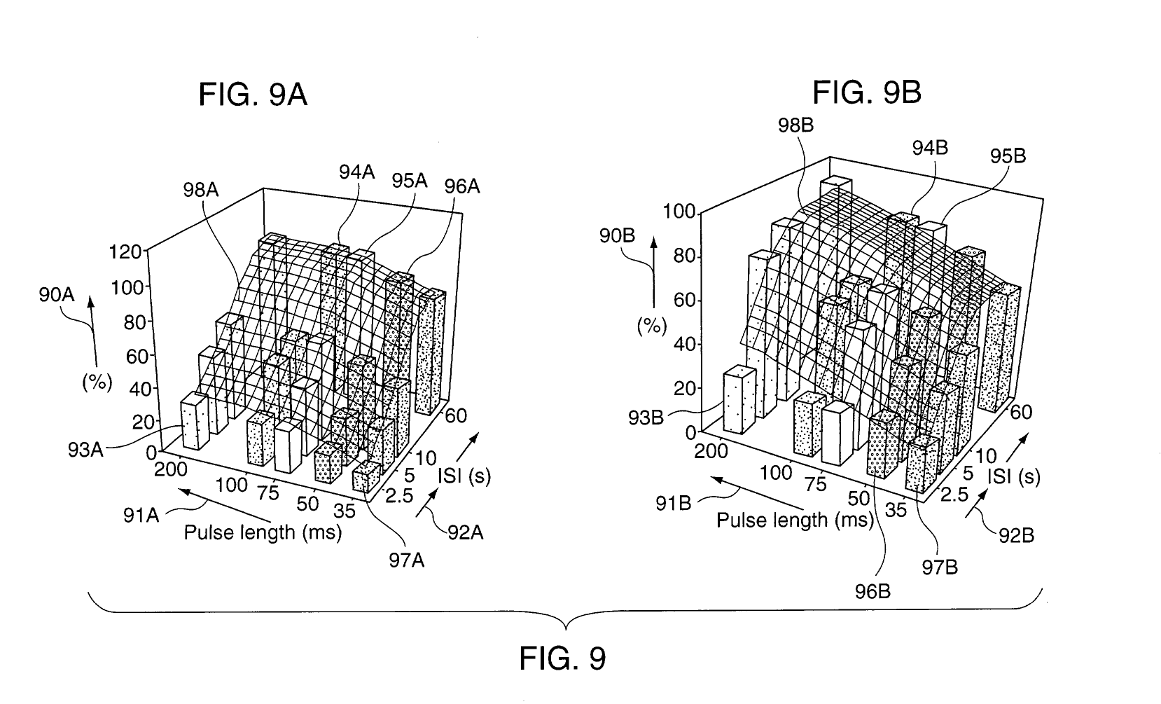

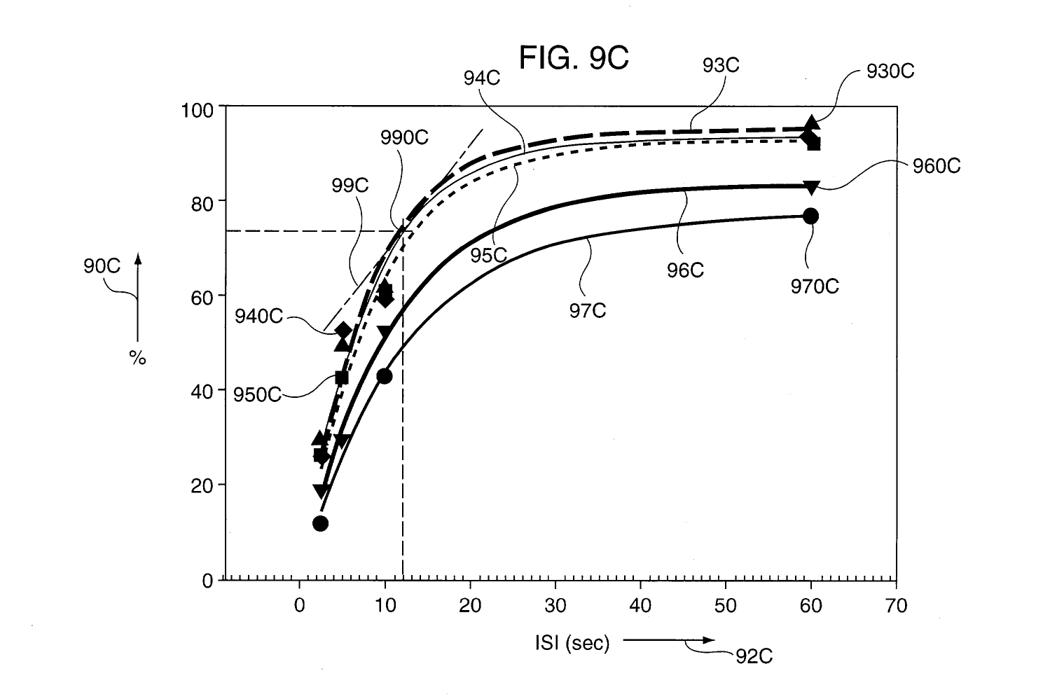

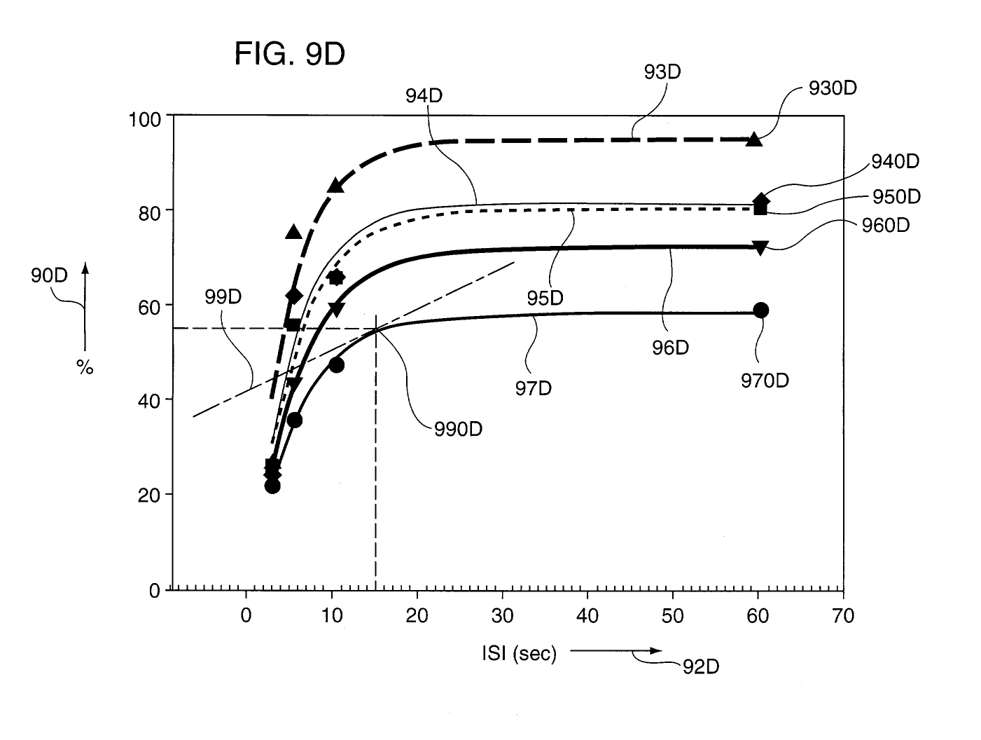

### DETAILED DESCRIPTION OF FIGS. 9A, 9B, 9C and 9D

FIGS. 9A, 9B, 9C and 9D are herein utilized to aid in interpreting the meanings of the parameters: Zₒ, A, B, C and D in the model:

Z=Zₒ+A(1−B^{X})+C(1−D^{Y})

wherein Z is the percentage of odour pulses correctly identified;

Y is the pulse duration, or “pulse length” measured in units of microseconds; and

X is the inter-stimulus interval, “ISI”, measured in units of seconds.

FIG. 9A is a dose-stimulus-frequency set of bar graphs for the malodour, n-butyric acid shown in three dimensions using the “X” axis, indicated by reference numeral 92A, the “Y” axis, indicated by reference numeral 91A and the “Z” axis, indicated by reference numeral 90A. The set of bar graphs indicated by reference numeral 97A is for a pulse length of 35 microseconds. The set of bar graphs indicated by reference numeral 96A is for a pulse length of 50 microseconds. The set of bar graphs indicated by reference numeral 95A is for a pulse length of 75 microseconds. The set of bar graphs indicated by reference numeral 94A is for a pulse length of 100 microseconds. The set of bar graphs indicated by reference numeral 93A is for a pulse length of 200 microseconds. The curved surface setting forth the relationship of the variables, X, Y and Z is indicated by reference numeral 98A.

FIG. 9B is a dose-stimulus-frequency set of bar graphs for the “pleasant” odour, amyl acetate, shown in three dimensions using the “X” axis, indicated by reference numeral 92B, the “Y” axis, indicated by reference numeral 91B and the “Z” axis, indicated by reference numeral 90B. The set of bar graphs indicated by reference numeral 97B is for a pulse length of 35 microseconds. The set of bar graphs indicated by reference numeral 96B is for a pulse length of 50 microseconds. The set of bar graphs indicated by reference numeral 95B is for a pulse length of 75 microseconds. The set of bar graphs indicated by reference numeral 94B is for a pulse length of 100 microseconds. The set of bar graphs indicated by reference numeral 93B is for a pulse length of 200 microseconds. The curved surface setting forth the relationship of the variables, X, Y and Z is indicated by reference numeral 98B.

FIG. 9C is a set of adaptation curves for the malodour, n-butyric acid, with the vertical axis being the “Z” axis, indicated by reference numeral 90C and the horizontal axis being the “X” axis indicated by reference numeral 92C. The graph indicated by reference numeral 97C and data points therefor 970C is for a pulse length of 35 microseconds. The graph indicated by reference numeral 96C and data points therefor 960C is for a pulse length of 50 microseconds. The graph indicated by reference numeral 95C and data points therefor 950C is for a pulse length of 75 microseconds. The graph indicated by reference numeral 94C and data points therefor 940C is for a pulse length of 100 microseconds. The graph indicated by reference numeral 93C and data points therefor 930C is for a pulse length of 200 microseconds.

FIG. 9D is a set of adaptation curves for the “pleasant” odour, amyl acetate, with the vertical axis being the “Z” axis, indicated by reference numeral 90D and the horizontal axis being the “X” axis indicated by reference numeral 92D. The graph indicated by reference numeral 97D and data points therefor 970D is for a pulse length of 35 microseconds. The graph indicated by reference numeral 96D and data points therefor 960D is for a pulse length of 50 microseconds. The graph indicated by reference numeral 95D and data points therefor 950D is for a pulse length of 75 microseconds. The graph indicated by reference numeral 94D and data points therefor 940D is for a pulse length of 100 microseconds. The graph indicated by reference numeral 93D and data points therefor 930D is for a pulse length of 200 microseconds.

In relating the substance of FIGS. 9A and 9B, to the model:

Z=Zₒ+A(1−B^{X})+C(1−D^{Y})

the parameters C and D are measures of the effects of (i) pulse duration, Y measured in microseconds, “ms”, along the “Y” axes indicated by reference numerals 91A and 91B and (ii) interstimulus interval, “ISI” measured in seconds, “s”, along the “X” axes indicated by reference numerals 92A and 92B in FIGS. 9A and 9B, respectively, on (iii) the percentage of odour pulses correctly identified, Z, measured along the “Z” axes indicated by reference numerals 90A and 90B in FIGS. 9A and 9B, respectively and each of C and D is a measure of the angle of inclination, θ, of the normal to the tangent plane to the X-Y-Z surface, indicated by reference numerals 98A and 98B in FIGS. 9A and 9B, respectively at a point (X₁,Y₁,Z₁) wherein θ=arccos[|ΔF (X₁,Y₁,Z₁)·|k |/}∥ΔF (X₁,Y₁,Z₁)∥}].

In relating the substance of FIGS. 9C and 9D, to the model:

Z=Zₒ+A(1−B^{X})+C(1−D^{Y})

concerning the A and B parameters, each parameter defines the effects of the inter-stimulus interval, X, measured along the “X” axis indicated by reference numerals 92C X and 92D, respectively for FIGS. 9C and 9D, and stimulus frequency, φ, on the percentage of odour pulses correctly identified, Z, measured along the vertical axes 90C and 90D in FIGS. 9C and 9D, respectively, and each is a measure of the degree of adaptation to malodours and habituation as measured by the tangent slopes, 99C and 99D to X-Z curves 93C and 93D at given points, 990C (X₁=12, Z₁=75), Δ F(X₁, Z₁) in FIGS. 9C and 990D (X₁=15, Z₁=54), Δ F(X₁, Z₁) in FIG. 9D. Also, C is a concentration parameter and is a measure of the magnitude of the tangent slope to the Y-Z curve at a given point, (Y₁, Z₁), Δ F(Y₁, Z₁).

What is claimed is:

1. A mixture consisting essentially of 1-cyclohexylethan-1-yl butyrate having the structure: 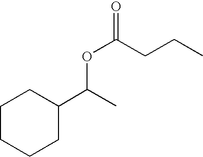
and 1-cyclohexylethan-1-yl acetate having the structure: 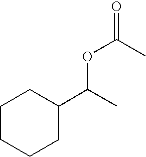
the weight ratio of 1-cyclohexylethan-1-yl butyrate:1-cyclohexylethan-1-yl acetate being from about 20:80 up to about 80:20, in the substantial absence of the compounds:
1-cyclohexylethan-1-ol having the structure: 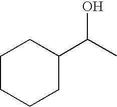
1-(4′-methylethyl)cyclohexylethan-1-yl propionate having the structure: 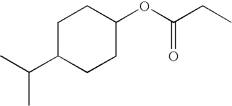
and 2′-hydroxy-1′-ethyl(2-phenoxy)acetate having the structure:

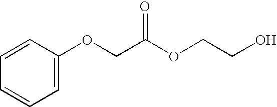

2. The mixture of claim 1 wherein the weight ratio of 1-cyclohexylethan-1-yl butyrate:1-cyclohexylethan-1-yl acetate is from about 50:50 up to about 80:20.

3. The mixture of 1-cyclohexylethan-1-yl butyrate having the structure: 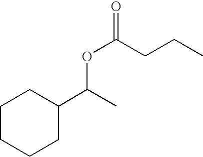
and 1-cyclohexylethan-1-yl acetate having the structure: 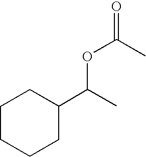
the weight ratio of 1-cyclohexylethan-1-yl butyrate:1-cyclohexylethan-1-yl acetate being from about 20:80 up to about 80:20 in the substantial absence of any additional fragrance substances or malodour counteractant substances.

4. The mixture of claim 3 wherein the weight ratio of 1-cyclohexylethan-1-yl butyrate: 1-cyclohexylethan-1-yl acetate is from about 50:50 up to about 80:20.

5. A process for counteracting a malodour emanating from a solid or liquid malodourous source into a 3-space proximate said source comprising the step of introducing into the 3-space proximate said source a synergistically-effective malodour-counteracting quantity and concentration of the composition of claim 1 as a single dose, as a continuous dose over a malodour-counteracting period of time, or as periodic doses over a malodour-counteracting period of time whereby the perceived total malodour intensity is substantially reduced or eliminated.

6. A process for counteracting a malodour present in a defined air 3-space comprising the step of introducing into said defined air 3-space a synergistically-effective malodour counteracting quantity and concentration of the composition of claim 1 as a single dose, as a continuous dose over a malodour-counteracting period of time, or as periodic doses over a malodour-counteracting period of time whereby the perceived total malodour intensity is substantially reduced or eliminated.

7. A process for counteracting a malodour emanating from a fragrance-containing and fragrance-evolving solid or liquid malodourous source into a 3-space proximate said source comprising the step of introducing into the 3-space proximate said source a synergistically-effective malodour-counteracting quantity and concentration of the composition of claim 1 as a single dose, as a continuous dose over a malodour-counteracting period of time, or as periodic doses over a malodour-counteracting period of time whereby the perceived total malodour intensity is substantially reduced or eliminated and the perceived odor intensity of the fragrance evolved into said 3-space from said source is substantially maintained.

8. A process for counteracting a malodour present in a defined fragrance-containing air 3-space comprising the step of introducing into said defined 3-space a synergistically-effective malodour-counteracting quantity and concentration of the composition of claim 1 as a single dose, as a continuous dose over a malodour-counteracting period of time, or as periodic doses over a malodour-counteracting period of time whereby the perceived total malodour intensity in said defined air 3-space is substantially reduced or eliminated and the perceived odor intensity of the fragrance existant in said defined air 3-space is substantially maintained.

9. A process for counteracting a malodour emanating from a solid or liquid malodourous source into a 3-space proximate said source comprising the step of introducing into the 3-space proximate said source a synergistically-effective malodour-counteracting quantity and concentration of the composition of claim 2 as a single dose, as a continuous dose over a malodour-counteracting period of time, or as periodic doses over a malodour-counteracting period of time whereby the perceived total malodour intensity is substantially reduced or eliminated.

10. A process for counteracting a malodour present in a defined air 3-space comprising the step of introducing into said defined air 3-space a synergistically-effective malodour counteracting quantity and concentration of the composition of claim 2 as a single dose, as a continuous dose over a malodour-counteracting period of time, or as periodic doses over a malodour-counteracting period of time whereby the perceived total malodour intensity is substantially reduced or eliminated.

11. A process for counteracting a malodour emanating from a fragrance-containing and fragrance-evolving solid or liquid malodourous source into a 3-space proximate said source comprising the step of introducing into the 3-space proximate said source a synergistically-effective malodour-counteracting quantity and concentration of the composition of claim 2 as a single dose, as a continuous dose over a malodour-counteracting period of time, or as periodic doses over a malodour-counteracting period of time whereby the perceived total malodour intensity is substantially reduced or eliminated and the perceived odor intensity of the fragrance evolved into said 3-space from said source is substantially maintained.

12. A process for counteracting a malodour present in a defined fragrance-containing air 3-space comprising the step of introducing into said defined 3-space a synergistically-effective malodour-counteracting quantity and concentration of the composition of claim 2 as a single dose, as a continuous dose over a malodour-counteracting period of time, or as periodic doses over a malodour-counteracting period of time whereby the perceived total malodour intensity in said defined air 3-space is substantially reduced or eliminated and the perceived odor intensity of the fragrance existant in said defined air 3-space is substantially maintained.

13. The process of claim 7 wherein the solid or liquid malodourous source evolving the malodour is selected from the group consisting of:
i. a malodourous herbicide;
ii. a malodourous antiviral composition;
iii. a malodourous fungicide;
iv. a malodourous bactericide;
v. a malodourous parasiticide;
vi. a malodourous insecticide;
vii. a malodourous depilatory preparation;
viii. a malodourous bleach composition;
ix. a malodourous hard surface-cleaning preparation;
x. a malodourous skin cleansing composition;
xi. a malodourous anti-microbial nail preparation;
xii. a malodourous hair setting composition;
xiii. a malodourous hair conditioning composition;
xiv. a malodourous trichological lotion;
xv. a malodourous skin lightening composition;
xvi. a malodourous detergent composition;
xvii. a malodourous soap composition;
xviii. a malodourous sunscreen composition;
xix. a malodourous fabric stain removal composition;
xx. a malodourous fabric conditioning composition;
xxi. a malodourous fabric anti-wrinkle composition;
xxii. a malodourous steam iron aroma composition;
xxiii. a malodourous candle composition;
xxiv. a malodourous plant growth regulating composition;
xxv. a malodourous plant growth stimulating composition;
xxvi. a malodourous fertilizer composition;
xxvii. a malodourous insect attractant composition;
xxviii. a malodourous insect repelling composition;
xxix. a malodourous drain cleaning composition;
xxx. a malodourous molluskicide composition;
xxxi. an anti-perspirant composition;
xxxii. a body deodorant composition;
xxxiii. a body deodorant/anti-perspirant device;
xxxiv. an air freshener device and
xxxv. an air freshener composition.

14. The process of claim 11 wherein the solid or liquid malodourous source evolving the malodour is selected from the group consisting of:
i. a malodourous herbicide;
ii. a malodourous antiviral composition;
iii. a malodourous fungicide;
iv. a malodourous bactericide;
v. a malodourous parasiticide;
vi. a malodourous insecticide;
vii. a malodourous depilatory preparation;
viii. a malodourous bleach composition;
ix. a malodourous hard surface-cleaning preparation;
x. a malodorous skin cleansing composition;
xi. a malodourous anti-microbial nail preparation;
xii. a malodourous hair setting composition;
xiii. a malodourous hair conditioning composition;
xiv. a malodourous trichological lotion;
xv. a malodourous skin lightening composition;
xvi. a malodourous detergent composition;
xvii. a malodourous soap composition;
xviii. a malodourous sunscreen composition;
xix. a malodourous fabric stain removal composition;
xx. a malodourous fabric anti-wrinkle composition;
xxi. a malodourous steam iron aroma composition;
xxii. a malodourous fabric conditioning composition;
xxiii. a malodourous candle composition;
xxiv. a malodourous plant growth regulating composition;
xxv. a malodourous plant growth stimulating composition;
xxvi. a malodourous fertilizer composition;
xxvii. a malodourous insect attractant composition;
xxviii. a malodourous insect repelling composition;
xxix. a malodourous drain cleaning composition;
xxx. a malodourous molluskicide composition;
xxxi. an antiperspirant composition;
xxxii. a body deodorant composition;
xxxiii. an anti-perspirant/body deodorant device;
xxxiv. an air freshener device and
xxxv. an air freshener composition.

15. The method of claim 13 wherein the malodour is caused by a malodour-causing quantity and concentration of at least one compound selected from the group consisting of aliphatic halohydrins, aliphatic amines, aliphatic N-oxides, dialkylamines, cycloaliphatic amines, cycloaliphatic N-oxides, cyclo-olefinic amines, cyclo-olefinic N-oxides, cycloaromatic amines, cycloaromatic N-oxides, hydroxyalkylamines, imine compounds, amide compounds, amino acids, polypeptides, modified antimicrobial proteins, diureides, nitriles, aliphatic mercaptans, cycloaliphatic mercaptans, mercaptoalkanoic acids, mercaptoalkanoic acid esters, aliphatic monosulfides, disulfides, trisulfides, sulfur oxides, sulfones and sultones, cycloaliphatic monosulfides, disulfides, trisulfides, sulfur oxides, sulfones and sultones, cyclo-olefinic monosulfides, disulfides, trisulfides, sulfur oxides, sulfones and sultones, cycloaromatic monosulfides, disulfides, trisulfides, sulfur oxides, sulfones and sultones, isothiocyanates, thiocyanates, dithiocyanates, isothiazolones, isothiazolinones, thiodiazinethiones, halosulfamates, aryl sulfonamides, lower aliphatic carboxylic acids, phenols, phosphines, aliphatic phosphites and phosphonates, cycloaliphatic phosphites and phosphonates, arsines, lower alcohols, lower ketones, hops, hops acids, aryl pyrazoles, oxazolines, isocyanurates, biguanides, extracts of krameria, hydantoins, pyrollidones, pyrollidone carboxylic acids, pyrollidone carboxylic acid esters, nitrophenols, N-substituted aspartic acids and pyrethroids.

16. The method of claim 14 wherein the malodour is caused by a malodour-causing quantity and concentration of at least one compound selected from the group consisting of aliphatic halohydrins, aliphatic amines, aliphatic N-oxides, dialkylamines, cycloaliphatic amines, cycloaliphatic N-oxides, cyclo-olefinic amines, cyclo-olefinic N-oxides, cycloaromatic amines, cycloaromatic N-oxides, hydroxyalkylamines, imine compounds, amide compounds, amino acids, polypeptides, modified antimicrobial proteins, diureides, nitriles, aliphatic mercaptans, cycloaliphatic mercaptans, mercaptoalkanoic acids, mercaptoalkanoic acid esters, aliphatic monosulfides, disulfides, trisulfides, sulfur oxides, sulfones and sultones, cycloaliphatic monosulfides, disulfides, trisulfides, sulfur oxides, sulfones and sultones, cyclo-olefinic monosulfides, disulfides, trisulfides, sulfur oxides, sulfones and sultones, cycloaromatic monosulfides, disulfides, trisulfides, sulfur oxides, sulfones and sultones, isothiocyanates, thiocyanates, dithiocyanates, isothiazolones, isothiazolinones, thiodiazinethiones, halosulfamates, aryl sulfonamides, lower aliphatic carboxylic acids, phenols, phosphines, aliphatic phosphites and phosphonates, cycloaliphatic phosphites and phosphonates, arsines, lower alcohols, lower ketones, hops, hops acids, aryl pyrazoles, oxazolines, isocyanurates, biguanides, extracts of krameria, hydantoins, pyrollidones, pyrollidone carboxylic acids, pyrollidone carboxylic acid esters, nitrophenols, N-substituted aspartic acids and pyrethroids.

17. A method for counteracting a malodour in a solid or liquid fragrance-containing soap or detergent caused by a malodour-causing quantity and concentration of at least one compound selected from the group consisting of aliphatic halohydrins, aliphatic amines, aliphatic N-oxides, dialkylamines, cycloaliphatic amines, cycloaliphatic N-oxides, cyclo-olefinic amines, cyclo-olefinic N-oxides, cycloaromatic amines, cycloaromatic N-oxides, hydroxyalkylamines, imine compounds, amide compounds, amino acids, polypeptides, modified antimicrobial proteins, diureides, nitriles, aliphatic mercaptans, cycloaliphatic mercaptans, mercaptoalkanoic acids, mercaptoalkanoic acid esters, aliphatic monosulfides, disulfides, trisulfides, sulfur oxides, sulfones and sultones, cycloaliphatic monosulfides, disulfides, trisulfides, sulfur oxides, sulfones and sultones, cyclo-olefinic monosulfides, disulfides, trisulfides, sulfur oxides, sulfones and sultones, cycloaromatic monosulfides, disulfides, trisulfides, sulfur oxides, sulfones and sultones, isothiocyanates, thiocyanates, dithiocyanates, isothiazolones, isothiazolinones, thiodiazinethiones, halosulfamates, aryl sulfonamides, lower aliphatic carboxylic acids, phenols, phosphines, aliphatic phosphites and phosphonates, cycloaliphatic phosphites and phosphonates, arsines, lower alcohols, lower ketones, hops, hops acids, aryl pyrazoles, oxazolines, isocyanurates, biguanides, extracts of krameria, hydantoins, pyrollidones, pyrollidone carboxylic acids, pyrollidone carboxylic acid esters, nitrophenols, N-substituted aspartic acids and pyrethroids comprising the step of introducing into the solid or liquid soap or detergent an effective malodour counteracting quantity and concentration of the composition of claim 1 whereby the perceived total malodour intensity in the solid or liquid soap or detergent is reduced or eliminated, and the perceived odor intensity of the fragrance contained in the solid or liquid soap or detergent is substantially maintained.

18. A method for counteracting a malodour in a solid or liquid fragrance-containing soap or detergent caused by a malodour-causing quantity and concentration of at least one compound selected from the group consisting of aliphatic halohydrins, aliphatic amines, aliphatic N-oxides, dialkylamines, cycloaliphatic amines, cycloaliphatic N-oxides, cyclo-olefinic amines, cyclo-olefinic N-oxides, cycloaromatic amines, cycloaromatic N-oxides, hydroxyalkylamines, imine compounds, amide compounds, amino acids, polypeptides, modified antimicrobial proteins, diureides, nitriles, aliphatic mercaptans, cycloaliphatic mercaptans, mercaptoalkanoic acids, mercaptoalkanoic acid esters, aliphatic monosulfides, disulfides, trisulfides, sulfur oxides, sulfones and sultones, cycloaliphatic monosulfides, disulfides, trisulfides, sulfur oxides, sulfones and sultones, cyclo-olefinic monosulfides, disulfides, trisulfides, sulfur oxides, sulfones and sultones, cycloaromatic monosulfides, disulfides, trisulfides, sulfur oxides, sulfones and sultones, isothiocyanates, thiocyanates, dithiocyanates, isothiazolones, isothiazolinones, thiodiazinethiones, halosulfamates, aryl sulfonamides, lower aliphatic carboxylic acids, phenols, phosphines, aliphatic phosphites and phosphonates, cycloaliphatic phosphites and phosphonates, arsines, lower alcohols, lower ketones, hops, hops acids, aryl pyrazoles, oxazolines, isocyanurates, biguanides, extracts of krameria, hydantoins, pyrollidones, pyrollidone carboxylic acids, pyrollidone carboxylic acid esters, nitrophenols, N-substituted aspartic acids and pyrethroids comprising the step of introducing into the solid or liquid soap or detergent an effective malodour counteracting quantity and concentration of the composition of claim 2 whereby the perceived total malodour intensity in the solid or liquid soap or detergent is reduced or eliminated, and the perceived odor intensity of the fragrance contained in the solid or liquid soap or detergent is substantially maintained.

19. A method of counteracting a malodour in a fragranced air 3-space caused by a malodour-causing quantity and concentration of at least one compound selected from the group consisting of aliphatic halohydrins, aliphatic amines, aliphatic N-oxides, dialkylamines, cycloaliphatic amines, cycloaliphatic N-oxides, cyclo-olefinic amines, cyclo-olefinic N-oxides, cycloaromatic amines, cycloaromatic N-oxides, hydroxyalkylamines, imine compounds, amide compounds, amino acids, polypeptides, modified antimicrobial proteins, diureides, nitriles, aliphatic mercaptans, cycloaliphatic mercaptans, mercaptoalkanoic acids, mercaptoalkanoic acid esters, aliphatic monosulfides, disulfides, trisulfides, sulfur oxides, sulfones and sultones, cycloaliphatic monosulfides, disulfides, trisulfides, sulfur oxides, sulfones and sultones, cyclo-olefinic monosulfides, disulfides, trisulfides, sulfur oxides, sulfones and sultones, cycloaromatic monosulfides, disulfides, trisulfides, sulfur oxides, sulfones and sultones, isothiocyanates, thiocyanates, dithiocyanates, isothiazolones, isothiazolinones, thiodiazinethiones, halosulfamates, aryl sulfonamides, lower aliphatic carboxylic acids, phenols, phosphines, aliphatic phosphites and phosphonates, cycloaliphatic phosphites and phosphonates, arsines, lower alcohols, lower ketones, hops, hops acids, aryl pyrazoles, oxazolines, isocyanurates, biguanides, extracts of krameria, hydantoins, pyrollidones, pyrollidone carboxylic acids, pyrollidone carboxylic acid esters, nitrophenols, N-substuted aspartic acids and pyrethroids comprising the step of introducing into said fragranced air 3-space an effective malodour counteracting quantity and concentration of the composition of claim 1 whereby the perceived total malodour intensity in the fragranced air 3-space is substantially reduced or eliminated, and the perceived odor intensity of the fragrance contained in the fragranced air 3-space is substantially maintained.

20. A method of counteracting a malodour in a fragranced air 3-space caused by a malodour-causing quantity and concentration of at least one compound selected from the group consisting of aliphatic halohydrins, aliphatic amines, aliphatic N-oxides, dialkylamines, cycloaliphatic amines, cycloaliphatic N-oxides, cyclo-olefinic amines, cyclo-olefinic N-oxides, cycloaromatic amines, cycloaromatic N-oxides, hydroxyalkylamines, imine compounds, amide compounds, amino acids, polypeptides, modified antimicrobial proteins, diureides, nitriles, aliphatic mercaptans, cycloaliphatic mercaptans, mercaptoalkanoic acids, mercaptoalkanoic acid esters, aliphatic monosulfides, disulfides, trisulfides, sulfur oxides, sulfones and sultones, cycloaliphatic monosulfides, disulfides, trisulfides, sulfur oxides, sulfones and sultones, cyclo-olefinic monosulfides, disulfides, trisulfides, sulfur oxides, sulfones and sultones, cycloaromatic monosulfides, disulfides, trisulfides, sulfur oxides, sulfones and sultones, isothiocyanates, thiocyanates, dithiocyanates, isothiazolones, isothiazolinones, thiodiazinethiones, halosulfamates, aryl sulfonamides, lower aliphatic carboxylic acids, phenols, phosphines, aliphatic phosphites and phosphonates, cycloaliphatic phosphites and phosphonates, arsines, lower alcohols, lower ketones, hops, hops acids, aryl pyrazoles, oxazolines, isocyanurates, biguanides, extracts of krameria, hydantoins, pyrollidones, pyrollidone carboxylic acids, pyrollidone carboxylic acid esters, nitrophenols, N-substuted aspartic acids and pyrethroids comprising the step of introducing into said fragranced air 3-space an effective malodour counteracting quantity and concentration of the composition of claim 2 whereby the perceived total malodour intensity in the fragranced air 3-space is substantially reduced or eliminated, and the perceived odor intensity of the fragrance contained in the fragranced air 3-space is substantially maintained.

21. The process of claim 5 wherein the malodour-counteracting composition is introduced into said 3-space as periodic doses over a malodour-counteracting period of time and is governed by the model:
Z=Zₒ+A(1−B^{X})+C(1−D^{Y})
wherein Z is the percentage of odour pulses correctly identified;
Y is the pulse duration measured in units of time;
X is the inter-stimulus interval measured in units of time;
Zₒ is a measure, as a scaled value of from −100 up to 100 of the sensitivity of the subjects to the odour detected;
A and B are parameters, each defining the effects of the inter-stimulus interval, X, and stimulus frequency, φ, on the percentage of odour pulses correctly identified, Z, and each is a measure of the degree of adaptation to malodours and habituation as measured by the tangent slope to the X-Z curve at a given point, (X₁, Z₁), Δ F(X₁, Z₁);
C is a concentration parameter and is a measure of the magnitude of the tangent slope to the Y-Z curve at a given point, (Y₁, Z₁), Δ F(Y₁, Z₁);
C and D are parameters, each being measures of the effects of pulse duration, Y, and inter-stimulus interval X on the percentage of odour pulses correctly identified, Z, and each is a measure of the angle of inclination,θ, of the normal to the tangent plane to the X-Y-Z surface at a point (X₁,Y₁,Z₁) wherein θ=arccos[|ΔF (X₁,Y₁,Z₁)·|k|/{{μΔF (X₁,Y₁,Z₁)∥}] with Zₒ being in the range of from about 45 up to about −15; A being in the range of from about 50 up to about 65; B being in the range of from about 0.93 up to about 0.97; C being in the range of from about 68 up to about 71; D being in the range of from about 0.98 up to about 0.99 and |k representing the “z” axis vector.

22. The process of claim 9 wherein the malodour-counteracting composition is introduced into said 3-space as periodic doses over a malodour-counteracting period of time, and is governed by the model:
Z=Z₎+A(1−B^{X})+C(1−D^{Y})
wherein Z is the percentage of odour pulses correctly identified;
Y is the pulse duration measured in units of time;
X is the inter-stimulus interval measured in units of time;
Zₒ is a measure, as a scaled value of from −100 up to 100 of the sensitivity of the subjects to the odour detected;
A and B are parameters, each defining the effects of the inter-stimulus interval, X, and stimulus frequency, φ, on the percentage of odour pulses correctly identified, Z, and each is a measure of the degree of adaptation to malodours and habituation as measured by the tangent slope to the X-Z curve at a given point, (X₁, Z₁), Δ F(X₁, Z₁);
C is a concentration parameter and is a measure of the magnitude of the tangent slope to the Y-Z curve at a given point, (Y₁, Z₁), Δ F(Y₁, Z₁);
C and D are parameters, each being measures of the effects of pulse duration, Y, and inter-stimulus interval X on the percentage of odour pulses correctly identified, Z, and each is a measure of the angle of inclination, θ, of the normal to the tangent plane to the X-Y-Z surface at a point (X₁,Y₁,Z₁) wherein θ=arccos [|ΔF (X₁,Y₁,Z₁)·|k|/{∥ΔF (X₁,Y₁,Z₁)∥}] with Zₒ being in the range of from about −45 up to about −25; A being in the range of from about 55 up to about 65; B being in the range of from about 0.95 up to about 0.97; C being in the range of from about 68 up to about 70; D being in the range of from about 0.98 up to about 0.99 and |k representing the “z” axis vector.

23. The process of claim 6 wherein the malodour-counteracting composition is introduced into said 3-space as periodic doses over a malodour-counteracting period of time and is governed by the model:
Z=Zₒ+A(1−B^{X})+C(1−D^{Y})
wherein Z is the percentage of odour pulses correctly identified;
Y is the pulse duration measured in units of time;
X is the inter-stimulus interval measured in units of time;
Zₒ is a measure, as a scaled value of from −100 up to 100 of the sensitivity of the subjects to the odour detected;
A and B are parameters, each defining the effects of the inter-stimulus interval, X, and stimulus frequency, φ, on the percentage of odour pulses correctly identified, Z, and each is a measure of the degree of adaptation to malodours and habituation as measured by the tangent slope to the X-Z curve at a given point, (X₁, Z₁), Δ F(X₁, Z₁);
C is a concentration parameter and is a measure of the magnitude of the tangent slope to the Y-Z curve at a given point, (Y₁, Z₁), Δ F(Y₁, Z₁);
C and D are parameters, each being measures of the effects of pulse duration, Y, and inter-stimulus interval X on the percentage of odour pulses correctly identified, Z, and each is a measure of the angle of inclinationsθ, of the normal to the tangent plane to the X-Y-Z surface at a point (X₁,Y₁,Z₁) wherein θ=arccos[|ΔF (X₁,Y₁,Z₁)·|k|/{∥ΔF (X₁,Y₁,Z₁)∥}] with Zₒ being in the range of from about −45 up to about −15; A being in the range of from about 50 up to about 65; B being in the range of from about 0.93 up to about 0.97; C being in the range of from about 68 up to about 71; D being in the range of from about 0.98 up to about 0.99 and |k representing the “z” axis vector.

24. The process of claim 10 wherein the malodour-counteracting composition is introduced into said 3-space as periodic doses over a malodour-counteracting period of time, and is governed by the model:
Z=Zₒ+A(1−B^{X})+C(1−D^{Y})
wherein Z is the percentage of odour pulses correctly identified;
Y is the pulse duration measured in units of time;
X is the inter-stimulus interval measured in units of time;
Zₒ is a measure, as a scaled value of from −100 up to 100 of the sensitivity of the subjects to the odour detected;
A and B are parameters, each defining the effects of the inter-stimulus interval, X, and stimulus frequency, φ, on the percentage of odour pulses correctly identified, Z, and each is a measure of the degree of adaptation to malodours and habituation as measured by the tangent slope to the X-Z curve at a given point, (X₁,Z₁), Δ F(X₁,Z₁);
C is a concentration parameter and is a measure of the magnitude of the tangent slope to the Y-Z curve at a given point, (Y₁, Z₁), Δ F(Y₁, Z₁);
C and D are parameters, each being measures of the effects of pulse duration, Y, and inter-stimulus interval X on the percentage of odour pulses correctly identified, Z, and each is a measure of the angle of inclination,θ, of the normal to the tangent plane to the X-Y-Z surface at a point (X₁,Y₁,Z₁) wherein θ=arccos [|ΔF (X₁,Y₁, Z₁)·|k |/{∥ΔF (X₁,Y₁,Z₁)∥}] with Zₒ being in the range of from about −45 up to about −25; A being in the range of from about 55 up to about 65; B being in the range of from about 0.95 up to about 0.97; C being in the range of from about 68 up to about 70; D being in the range of from about 0.98 up to about 0.99 and |k representing the “z” axis vector.

25. The process of claim 8 wherein the malodour-counteracting composition is introduced into said 3-space as periodic doses over a malodour-counteracting period of time and is governed by the model:
Z=Zₒ+A(1−B^{X})+C(1−D^{Y})
wherein Z is the percentage of odour pulses correctly identified;
Y is the pulse duration measured in units of time;
X is the inter-stimulus interval measured in units of time;
Zₒ is a measure, as a scaled value of from −100 up to 100 of the sensitivity of the subjects to the odour detected;
A and B are parameters, each defining the effects of the inter-stimulus interval, X, and stimulus frequency, φ, on the percentage of odour pulses correctly identified, Z, and each is a measure of the degree of adaptation to malodours and habituation as measured by the tangent slope to the X-Z curve at a given point, (X₁,Z₁), Δ F(X₁, Z₁);
C is a concentration parameter and is a measure of the magnitude of the tangent slope to the Y-Z curve at a given point, (Y₁, Z₁), Δ F(Y₁, Z₁);
C and D are parameters, each being measures of the effects of pulse duration, Y, and inter-stimulus interval X on the percentage of odour pulses correctly identified, Z, and each is a measure of the angle of inclinations,θ, of the normal to the tangent plane to the X-Y-Z surface at a point (X₁,Y₁,Z₁) wherein θ=arccos[|ΔF (X₁,Y₁,Z₁)·|k |/{∥ΔF (X₁,Y₁,Z₁)∥}] with Zₒ being in the range of from about −45 up to about −15; A being in the range of from about 50 up to about 65; B being in the range of from about 0.93 up to about 0.97; C being in the range of from about 68 up to about 71; D being in the range of from about 0.98 up to about 0.99 and |k representing the “z” axis vector.

26. The process of claim 11 wherein the malodour-counteracting composition is introduced into said 3-space as periodic doses over a malodour-counteracting period of time, and is governed by the model:
Z=Zₒ+A(1−B^{X})+C(1−D^{Y})
wherein Z is the percentage of odour pulses correctly identified;
Y is the pulse duration measured in units of time;
X is the inter-stimulus interval measured in units of time;
Zₒ is a measure, as a scaled value of from −100 up to 100 of the sensitivity of the subjects to the odour detected;
A and B are parameters, each defining the effects of the inter-stimulus interval, X, and stimulus frequency, φ, on the percentage of odour pulses correctly identified, Z, and each is a measure of the degree of adaptation to malodours and habituation as measured by the tangent slope to the X-Z curve at a given point, (X₁, Z₁), Δ F(X₁,Z₁);
C is a concentration parameter and is a measure of the magnitude of the tangent slope to the Y-Z curve at a given point, (Y₁, Z₁), Δ F(Y₁, Z₁);
C and D are parameters, each being measures of the effects of pulse duration, Y, and inter-stimulus interval X on the percentage of odour pulses correctly identified, Z, and each is a measure of the angle of inclinations,θ, of the normal to the tangent plane to the X-Y-Z surface at a point (X₁,Y₁,Z₁) wherein θ=arccos [|ΔF (X₁,Y₁,Z₁)·|k |/{∥ΔF (X₁,Y₁,Z₁)∥}] with Zₒ being in the range of from about −45 up to about −25; A being in the range of from about 55 up to about 65; B being in the range of from about 0.95 up to about 0.97; C being in the range of from about 68 up to about 70; D being in the range of from about 0.98 up to about 0.99 and |k representing the “z” axis vector.
